(19) United States
(12) Patent Application Publication
(10) Pub. No.: US 2010/0279863 A1
(43) Pub. Date: Nov. 4, 2010

(54) HERBICIDAL COMPOUNDS BASED ON N-AZINYL-N'-PYRIDYLSULFONYLUREAS

(75) Inventors: Klaus-Helmut MÜLLER, Dusseldorf (DE); Christian WALDRAFF, Bad Vilbel (DE); Ernst Rudolf GESING, Erkrath (DE); Guido BOJACK, Wiesbaden-Naurod (DE); Heinz KEHNE, Hofheim (DE); Christopher Hugh ROSINGER, Hofheim (DE); Dieter FEUCHT, Eschborn (DE)

Correspondence Address: Baker Donelson Bearman, Caldwell & Berkowitz, PC, 920 Massachusetts Ave, NW, Suite 900, Washington, DC, 20001, US

(73) Assignee: Bayer CropScience AG, Monheim (DE)

(21) Appl. No.: 12/769,400

(22) Filed: Apr. 28, 2010

(30) Foreign Application Priority Data
May 2, 2009 (EP) EP 09006045.0

(51) Int. Cl. A01N 43/88; C07D 413/14; C07D 413/04; C07D 498/04; A01N 43/66; A01N 25/32; A01P 13/00

(52) U.S. Cl. 504/105; 544/65; 504/130; 504/133

(57) ABSTRACT

There are described compounds of the formula (I)

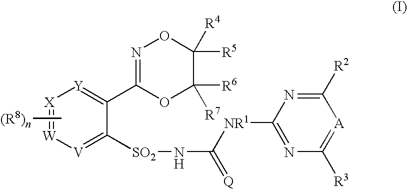

in which the respective substituents have the meanings given in the description. The compounds of the formula (I) can be used for example as herbicides and plant growth regulators.

### CROSS REFERENCE TO RELATED APPLICATIONS

This application claims priority to EP 09006045.0 filed May 2, 2009, the content of which is incorporated herein by reference in its entirety.

### BACKGROUND OF THE INVENTION

1. Field of the Invention

The present invention relates to N-azinyl-N′-pyridylsulfonylureas. The present invention furthermore relates to mixtures of the abovementioned urea derivatives with other herbicides and/or safeners. Moreover, the present invention relates to processes for the preparation of the abovementioned urea derivatives and to the use of these compounds as herbicides and plant growth regulators alone and in admixture with safeners and/or in admixture with other herbicides, in particular to their use for controlling plants in specific plant crops or as plant growth regulators.

2. Description of Related Art

It has already been disclosed that certain N-azinyl-N′-arylsulfonylureas with single open-chain hydroxamic ester groups in the aryl moiety such as, for example, N-(4,6-dimethylpyrimidin-2-yl)-N′-(2-methoxyaminocarbonylphenylsulfonyl)urea and the corresponding N′-(2-n-octyloxyaminocarbonylphenylsulfonyl)urea have herbicidal properties (cf. DE 3 516 435 A, EP 0 173 958 A, U.S. Pat. No. 4,704,158).

Furthermore, there are also known certain herbicidally active N-azinyl-N′-hetarylsulfonylureas which are substituted in the hetaryl moiety by O,N-dialkylated, likewise open-chain hydroxamic acid groups (cf. EP 0 301 784 A); however, corresponding cyclic hydroxamic acid derivatives have not been described to date.

Furthermore, U.S. Pat. No. 5,476,936 discloses herbicidal N-azinyl-N′-hetarylsulfonylureas of the formula

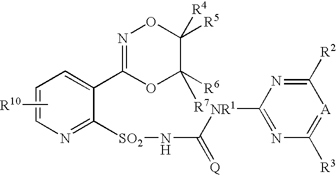

(where the individual radicals have the meanings given in U.S. Pat. No. 5,476,936). In these compounds, the sulfonylurea radical is in the α-position relative to the nitrogen atom of the pyridine ring.

The active substances which are already known from the above-mentioned specifications have disadvantages when used, be it
(a) that they have no or only else an insufficient herbicidal activity against harmful plants,
(b) that only an unduly narrow spectrum of harmful plants can be controlled, or
(c) that they have an unduly narrow selectivity in crops of useful plants.

### SUMMARY OF THE INVENTION

It is therefore desirable to provide alternative chemical active substances based on corresponding urea derivatives which can be employed as herbicides or plant growth regulators and with which certain advantages in comparison with the prior-art systems are associated.

As a result, the object of the present invention is in general to provide corresponding alternative urea derivatives which can be employed as herbicides or plant growth regulators, in particular with a satisfactory herbicidal activity against harmful plants, a broad spectrum for harmful plants and/or a high selectivity in crops of useful plants. In this context, these urea derivatives should preferably feature a better profile of characteristics, in particular a better herbicidal activity against harmful plants, a broader spectrum for harmful plants and/or a higher selectivity in crops of useful plants than the urea derivatives known from the prior art.

There have now been found novel N-azinyl-N′-pyridylsulfonylureas of the formula (I),

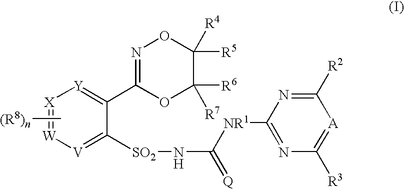

in which
W, X and Y are chosen such that one index of these indices represents a nitrogen atom and the remaining indices represent carbon atoms, the carbon atoms being unsubstituted or independently of one another substituted by R⁸;
V represents a carbon atom which is unsubstituted or substituted by R⁸;
A is selected from the group consisting of nitrogen and CR⁹;
   where
   R⁹ is selected from the group consisting of hydrogen, alkyl, halogen and haloalkyl;
R¹ is selected from the group consisting of hydrogen and an unsubstituted or substituted radical from the series consisting of alkyl, alkoxy, alkoxyalkyl, alkenyl, alkynyl, cycloalkyl, cycloalkylalkyl, aralkyl and aryl;
R² is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino;
R³ is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino,
R⁴ to R⁷, in each case independently of one another, are selected from the group consisting of hydrogen, halogen, cyano, alkyl, alkoxy, alkylthio, alkylsulfinyl, alkylsulfonyl, alkylamino, dialkylamino, alkylcarbonyl, alkoxycarbonyl, alkylaminocarbonyl or dialkylaminocarbonyl, it being possible for the radicals to be unsubstituted or to have attached to them one or more radicals selected from the group consisting of halogen, cyano, alkoxy and alkylthio, or R⁴ and R⁶, and R⁵ and R⁷, respectively, represent an alkylidene group which is optionally interrupted by oxygen or sulfur,
R⁸ is selected from the group consisting of halogen, cyano, thiocyanato, nitro, alkyl, alkoxy, alkylthio, alkylsulfinyl, alkylsulfonyl, alkylamino, dialkylamino, alkylcarbonyl, alkoxycarbonyl, alkylaminocarbonyl, or dialkylaminocarbonyl, it being possible for the radicals to be unsubstituted or to have attached to them one or more radicals selected from the group consisting of halogen, cyano, alkoxy and alkylthio,
Q is selected from the group consisting of oxygen or sulfur, in particular oxygen,
n is an integer from 0 to 3,
   and salts of compounds of the formula (I),
   with the proviso that, in those cases where n is 0, 1 or 2, the unsubstituted carbon atoms V, W, X and/or Y are saturated with hydrogen.

### DETAILED DESCRIPTION OF A PREFERRED EMBODIMENT

A first embodiment of the present invention comprises compounds of the formula (I) in which

A is preferably selected from the group consisting of nitrogen and CH.

A second embodiment of the present inventions comprises compounds of the formula (I) in which
R¹ is preferably selected from the group consisting of hydrogen, alkyl, alkoxy, alkoxyalkyl, alkenyl and alkynyl, it being possible for the radicals to be unsubstituted or to have attached to them one or more halogen atoms,
R¹ is especially preferably selected from the group consisting of hydrogen, methyl, ethyl, methoxy, methoxymethyl and ethoxy,
R¹ is particularly preferably selected from the group consisting of hydrogen and methyl,
   and
R¹ is specifically preferably hydrogen.

A third embodiment of the present invention comprises compounds of the formula (I) in which
R² is preferably selected from the group consisting of hydrogen, halogen, alkyl, alkoxy, alkylthio, alkylamino and dialkylamino, it being possible for the radicals to be unsubstituted or to have attached to them one or more halogen atoms,
R² is especially preferably selected from the group consisting of hydrogen, chlorine, methyl, ethyl, trifluoromethyl, methoxy, ethoxy, trifluoroethoxy, difluoromethoxy, methylthio, methylamino and dimethylamino,
   and
R² is specifically preferably selected from the group consisting of hydrogen, chlorine, methyl, methoxy, methylthio and dimethylamino.

A fourth embodiment of the present invention comprises compounds of the formula (I) in which
R³ is preferably selected from the group consisting of hydrogen, halogen, alkyl, alkoxy, alkylthio, alkylamino and dialkylamino, it being possible for the radicals to be unsubstituted or to have attached to them one or more halogen atoms,
R³ is especially preferably selected from the group consisting of hydrogen, chlorine, methyl, ethyl, trifluoromethyl, methoxy, ethoxy, trifluoroethoxy, difluoromethoxy, methylthio, methylamino and dimethylamino,
   and
R³ is especially preferably selected from the group consisting of methyl, methoxy and trifluoroethoxy.

A fifth embodiment of the present invention comprises compounds of the formula (I) in which
R⁴ to R⁷, in each case independently of one another, are preferably selected from the group consisting of hydrogen, halogen, cyano, alkyl, alkoxy, alkylthio, alkylsulfinyl, alkylsulfonyl, alkylamino, dialkylamino, alkylcarbonyl, alkoxycarbonyl, alkylaminocarbonyl or dialkylamino, it being possible for the radicals to be unsubstituted or to have attached to them one or more halogen atoms,
R⁴ to R⁷, in each case independently of one another, are especially preferably selected from the group consisting of hydrogen, fluorine, chlorine, cyano, methyl, ethyl, propyl, isopropyl, cyclopropyl, n-, s- or tert-butyl, methylthio, methylsulfinyl, methylsulfonyl, methoxycarbonyl and ethoxycarbonyl, it being possible for the radicals to be unsubstituted or to have attached to them one or more halogen atoms, and R⁴ and R⁶ and/or R⁵ and R⁷ represent a CH₂—CH₂—CH₂— group, a CH₂—CH₂—CH₂—CH₂— group, a CH₂—O—CH₂— group, a CH₂—S—CH₂— group, a CH₂—O—CH₂—CH₂— group, a CH₂—CH₂—O—CH₂— group or a CH₂—CH₂—O—CH₂—CH₂— group,
   and
R⁴ to R⁷, in each case independently of one another, are specifically preferably selected from the group consisting of hydrogen, methyl, ethyl, propyl and isopropyl, or R⁴ and R⁶ and/or R⁵ and R⁷ represent a CH₂—CH₂—CH₂— group, a CH₂—CH₂—CH₂—CH₂— group, a CH₂—O—CH₂— group, a CH₂—S—CH₂— group, a CH₂—O—CH₂—CH₂— group, a CH₂—CH₂—O—CH₂— group or a CH₂—CH₂—O—CH₂—CH₂— group.

A sixth embodiment of the present invention comprises compounds of the formula (I) in which
R⁸ is preferably selected from the group consisting of halogen, cyano, thiocyanato, nitro, alkyl, alkoxy, alkylthio, alkylsulfinyl, alkylsulfonyl, alkylamino, dialkylamino, alkylcarbonyl, alkoxycarbonyl, alkylaminocarbonyl and dialkylaminocarbonyl, it being possible for the radicals to be unsubstituted or to have attached to them one or more halogen atoms, and
R⁸ is especially preferably selected from the group consisting of fluorine, chlorine, bromine, iodine, cyano, methyl, methoxy, ethoxy, methylthio, ethylthio, methylsulfinyl, ethylsulfinyl, methylsulfonyl, ethylsulfonyl, methylamino and dimethylamino, it being possible for the radicals to be unsubstituted or to have attached to them one or more halogen atoms.

A seventh embodiment of the present invention comprises compounds of the formula (I) in which

n is 0 or 1, preferably n is 0.

If, within the scope of the present invention, a plurality of radicals R⁸ are present, that is to say n is 2 or 3, the radicals R⁸ may be identical or different.

Within the scope of these embodiments of the present invention, it is possible to combine the individual general, preferred and especially preferred meanings for the substituents R¹ to R⁸, Q and A and for the index n as desired. This means that the present invention comprises compounds of the formula (I) in which for example the substituent R¹ has a preferred meaning and the substituents R² to R⁸ have the general meanings, or else for example that the substituent R² has a preferred meaning, the substituent R³ an especially preferred meaning, and the remaining substituents have the general meanings. These individual combinations are not mentioned expressly for reasons of clarity, but must be considered as being comprised by the present invention.

The heterocycle in the compounds of the formula (I)

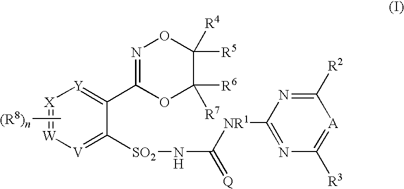

which is linked directly to the 2-(5,6-dihydro-[1,4,2]-dioxazin)-3-yl radical has a nitrogen atom, i.e. a radical selected from the group consisting of W, X and Y corresponds to a nitrogen atom. The remaining two radicals have the meaning of a carbon atom, it being possible for the carbon atoms optionally to be substituted by one or two radicals R⁸, which can be identical or different.

In a special embodiment of the present invention, the compound of the formula (I) therefore has the following structure (Ia), in which W has the meaning of a nitrogen atom:

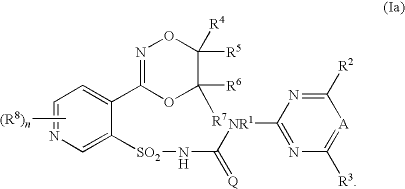

In yet a further embodiment of the present invention, the compound of the formula (I) therefore has the following structure (Ib), in which X has the meaning of a nitrogen atom:

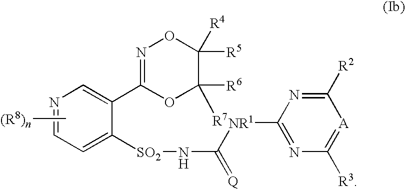

In yet a further embodiment of the present invention, the compound of the formula (I) therefore has the following structure (Ic), in which Y has the meaning of a nitrogen atom:

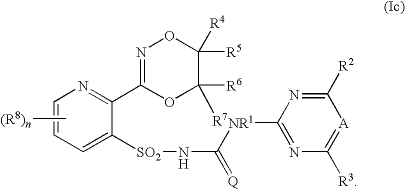

In each of these three embodiments, the individual radicals R¹ to R⁸, A and Q and the index n can have the above-defined general, preferred and especially preferred meanings.

Especially preferred among these three embodiments are according to the invention those compounds in which either W or Y has the meaning nitrogen (compounds of the formula (Ia) and (Ic)).

Particularly preferred are those compounds of the formula (I), in which W has the meaning nitrogen (compounds of the formula (Ia)).

In the compounds of the formula (I), the substituents and radicals R¹ to R⁸, Q and A and the index n have the above general, preferred, especially preferred, particularly preferred and very particularly preferred meanings.

The present invention preferably also relates to the lithium, sodium, potassium, magnesium, calcium, ammonium, C₁-C₄-alkylammonium, di(C₁-C₄-alkyl)ammonium, tri(C₁-C₄-alkyl)ammonium, tetra(C₁-C₄-alkyl)ammonium, tri(C₁-C₄-alkyl)sulfonium, C₅- or C₆-cycloalkylammonium, di(C₁-C₂-alkyl)benzylammonium and tri(C₁-C₂-alkyl)benzylammonium salts of compounds of the formula (I) in which R¹ to R⁸, A, Q and n have the above general, preferred, especially preferred and particularly preferred meanings and which can be prepared by generally customary methods.

In addition, the compounds of the formula (I) can where appropriate form salts by addition reaction of a suitable inorganic or organic acid, such as, for example, HCl, HBr, H₂SO₄ or HNO₃, but also oxalic acid or sulfonic acids, onto a basic group such as, for example, amino or alkylamino. Suitable substituents which are present in deprotonated form, such as, for example, sulfonic acids or carboxylic acids, can form internal salts with groups which can be protonated in turn, such as amino groups. Salts can also be formed by replacing, in the case of suitable substituents such as, for example, sulfonic acids or carboxylic acids, the hydrogen by a cation which is suitable for the agrochemical sector. Examples of these salts are metal salts, in particular alkali metal salts or alkaline earth metal salts, in particular sodium and potassium salts, or else ammonium salts, salts with organic amines or quaternary ammonium salts with cations of the formula [NRR′R″R′″]⁺, in which R to R′″ in each case independently of another represent an organic radical, in particular alkyl, aryl, aralkyl or alkylaryl.

In the formula (I) and all remaining formulae in the present invention, the radicals alkyl, alkoxy, haloalkyl, haloalkoxy, alkylamino, alkylthio, haloalkylthio, alkylsulfinyl, alkylsulfonyl, haloalkylsulfinyl and haloalkylsulfonyl and the corresponding unsaturated and/or substituted radicals in the carbon skeleton can in each case be straight-chain or branched. Unless otherwise specified, the lower carbon skeletons, for example those with 1 to 6 carbon atoms, in particular 1 to 4 carbon atoms, or, in the case of unsaturated groups, having 2 to 6 carbon atoms, in particular 2 to 4 carbon atoms, are preferred among these radicals. Alkyl radicals, also in the composite meanings such as alkoxy, haloalkyl and the like, are, for example, methyl, ethyl, propyls such as n- or i-propyl, butyls such as n-, iso- or tert-butyl, pentyls such as n-pentyl, isopentyl or neopentyl, hexyls such as n-hexyl, i-hexyl, 3-methylpentyl, 2,2-dimethylbutyl or 2,3-dimethylbutyl, heptyls such as n-heptyl, 1-methylhexyl or 1,4-dimethylpentyl; alkenyl and alkynyl radicals have the meaning of the unsaturated radicals which are possible and which correspond to the alkyl radicals and which comprise at least one double bond or triple bond, preferably one double bond or triple bond. Alkenyl is, for example, vinyl, allyl, 1-methylprop-2-en-1-yl, 2-methylprop-2-en-1-yl, but-2-en-1-yl, but-3-en-1-yl, 1-methylbut-3-en-1-yl and 1-methylbut-2-en-1-yl; alkynyl is, for example, ethynyl, propargyl, but-2-yn-1-yl, but-3-yn-1-yl and 1-methylbut-3-yn-1-yl.

Examples of cycloalkyl groups are cyclopropyl, cyclobutyl, cyclopentyl, cyclohexyl, cycloheptyl and cyclooctyl. The cycloalkyl groups may be present in bicyclic or tricyclic form.

If haloalkyl groups and haloalkyl radicals of haloalkoxy, haloalkylthio, haloalkenyl, haloalkynyl and the like are specified, the lower carbon skeletons, for example those having 1 to 6 carbon atoms or 2 to 6, in particular 1 to 4, carbon atoms or preferably 2 to 4 carbon atoms, and the corresponding unsaturated and/or substituted radicals in the carbon skeleton are in each case straight-chain or branched in these radicals. Examples are difluoromethyl, 2,2,2-trifluoroethyl, trifluoroallyl, 1-chloroprop-1-yl-3-yl. The term “halo” is used synonymously with “halogen” according to the invention.

Alkylene groups in these radicals are the lower carbon skeletons, for example those having 1 to 10 carbon atoms, in particular 1 to 6 carbon atoms or preferably 2 to 4 carbon atoms (unless otherwise defined) and the corresponding unsaturated and/or substituted radicals in the carbon skeleton which can in each case be straight-chain or branched. Examples are methylene, ethylene, n- and isopropylene and n-, sec-, iso- and tert-butylene.

Hydroxyalkyl groups in these radicals are the lower carbon skeletons, for example those having 1 to 6 carbon atoms, in particular 1 to 4 carbon atoms, and the corresponding unsaturated and/or substituted radicals in the carbon skeleton which can in each case be straight-chain or branched. Examples are 1,2-dihydroxyethyl and 3-hydroxypropyl.

Halogen is fluorine, chlorine, bromine or iodine. Haloalkyl, haloalkenyl and haloalkynyl are alkyl, alkenyl or alkynyl which are partially or fully substituted by halogen, preferably by fluorine, chlorine or bromine, in particular by fluorine and/or chlorine, for example monohaloalkyl, perhaloalkyl, CF₃, CHF₂, CH₂F, CF₃CF₂, CH₂FCHCl, CCl₃, CHCl₂, CH₂CH₂Cl; haloalkoxy is, for example, OCF₃, OCHF₂, OCH₂F, CF₃CF₂O, OCH₂CF₃ and OCH₂CH₂Cl; the same applies analogously to haloalkenyl and other halogen-substituted radicals. The radical halogen with the above meaning (fluorine, chlorine, bromine or iodine) is also abbreviated to Hal hereinbelow.

Aryl is a mono-, bi- or polycyclic aromatic system, for example phenyl or naphthyl, preferably phenyl.

Unless otherwise defined, the definition “substituted by one or more radicals” means one or more identical or different radicals.

The substituents mentioned by way of example (“first substituent level”) can, if they contain hydrocarbon-comprising moieties, optionally be further substituted therein (“second substituent level”), for example by one of the substituents as defined for the first substituent level. Corresponding further substituent levels are possible. Preferably, the term “substituted radical” only comprises one or two substituent levels.

Preferred in the case of radicals with carbon atoms are those with 1 to 6 carbon atoms, preferably 1 to 4 carbon atoms, in particular 1 or 2 carbon atoms. As a rule, preferred are substituents from the group consisting of halogen, for example fluorine and chlorine, (C₁-C₄)-alkyl, preferably methyl or ethyl, (C₁-C₄)-haloalkyl, preferably trifluoromethyl, (C₁-C₄)-alkoxy, preferably methoxy or ethoxy, (C₁-C₄)-haloalkoxy, nitro and cyano.

When an aryl radical is substituted, this may preferably be phenyl which is monosubstituted or polysubstituted, preferably up to trisubstituted, by identical or different radicals selected from the group consisting of halogen, (C₁-C₄)-alkyl, (C₁-C₄)-alkoxy, (C₁-C₄)-haloalkyl, (C₁-C₄)-haloalkoxy, cyano and nitro, for example o-, m- and p-tolyl, dimethylphenyls, 2-, 3- and 4-chlorophenyl, 2-, 3- and 4-trifluoromethyl and 2-, 3- and 4-trichloromethylphenyl, 2,4-, 3,5-, 2,5- and 2,3-dichlorophenyl, o-, m- and p-methoxyphenyl.

Depending on the nature and linkage of the substituents, the compounds of the formula (I) may be present as stereoisomers. The stereoisomers which are possible, such as enantiomers, diastereomers, Z and E isomers, which are defined by their specific spatial shape, are all comprised by formula (I).

If, for example, one or more alkenyl groups are present, then diastereomers (Z and E isomers) may occur. If, for example, one or more asymmetric carbon atoms are present, then enantiomers and diastereomers may occur. Stereoisomers may be obtained by customary separation methods from the mixtures which the preparation gives rise to. Likewise, stereoisomers may be prepared selectively by using stereoselective reactions, using optically active starting materials and/or adjuvants. The invention therefore also relates to all stereoisomers which are comprised by the formula (II), but not specified in their specific stereometric form, and to mixtures of these.

### Preparation of the Compounds of the Formula (I) According to the Invention

The present invention furthermore relates to processes for the preparation of corresponding compounds of the formula (I) and/or their salts.

In a first embodiment of the present invention, the compounds of the formula (I) are prepared by reacting (5,6-dihydro-1,4,2-dioxazin-3-yl)pyridylsulfonamides of the formula (II)

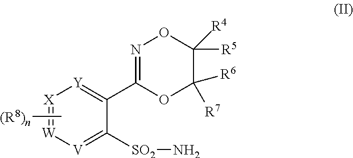

with a heterocyclic (thio)carbamate of the formula (III)

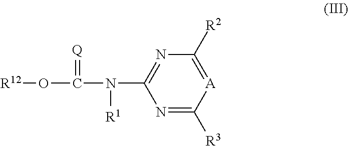

in which R¹² is a substituted or unsubstituted (C₁-C₂₀)-hydrocarbon radical such as aryl or alkyl, preferably optionally substituted phenyl or optionally substituted (C₁-C₄)-alkyl, and in which V, W, X, Y, R¹ to R⁸, Q, A and n have the above-mentioned meanings.

In this context, the compounds of the formula (II) can be obtained by reacting the compounds of the formula (X) with a chlorinating agent such as chlorine gas and ammonia solution as shown in the reaction scheme hereinbelow (where the radicals have the above meaning):

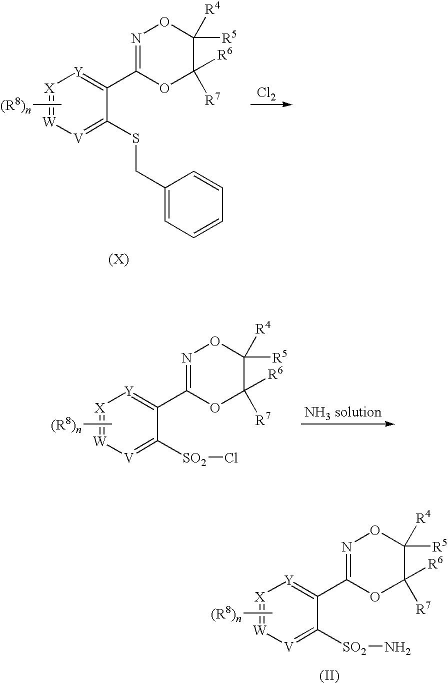

The compounds of the formula (X), in turn, can be obtained by reacting compounds of the formula (XI) with optionally substituted ethane derivatives of the formula (XII) as shown in the reaction scheme hereinbelow (where the radicals have the abovementioned meanings):

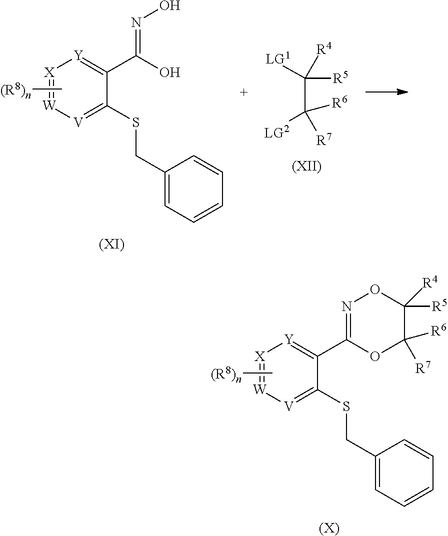

In the formula (XII), LG¹ and LG² are a leaving group, for example halide or sulfonate, it being possible for the radicals LG¹ and LG² to be identical or different.

The compounds of the formula (XI), in turn, can be obtained by reacting carboxylic esters, for example methyl esters, with hydroxylamine, starting from compounds of the formula (XIII) as shown in the reaction scheme hereinbelow (where the radicals have the abovementioned meanings):

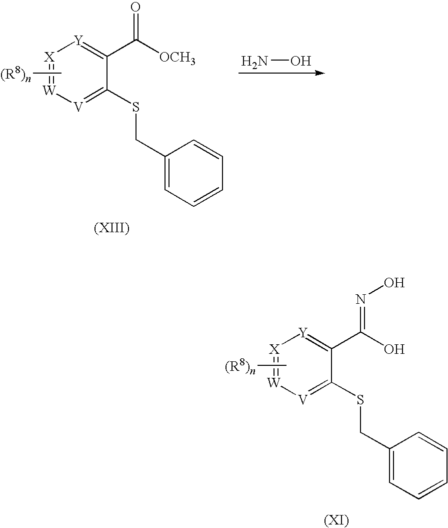

The compounds of the formula (XIII), in turn, can be obtained by reacting carboxylic acids of the formula (XIV) with thionyl chloride and alcohols, for example methanol, starting from compounds of the formula (XIV) as shown in the reaction scheme hereinbelow (where the radicals have the abovementioned meanings):

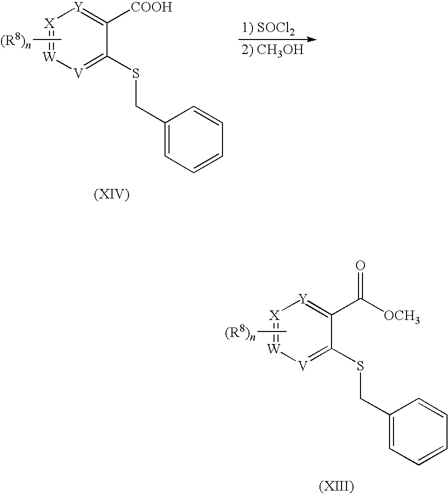

(Benzylsulfanyl)pyridinecarboxylic acids of the formula (XIV) are known from the prior art (cf. U.S. Pat. No. 4,767,766, J. Med. Chem. 1974, 17(10), 1065-1071 and DE 2,216,576) and can be prepared by methods known to the skilled worker from commercially available precursors (for example 3-chloroisonicotinonitrile, 4-chloronicotinic acid, 3-sulfanylpyridine-2-carboxylic acid).

In a second embodiment of the present invention, the compounds of the formula (I) are prepared by reacting 5,6-dihydro-1,4,2-dioxazin-3-ylpyridinesulfonyl iso(thio)cyanates of the formula (IV)

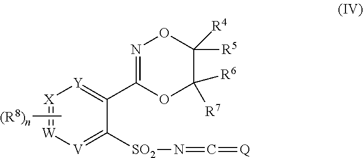

(the preparation of corresponding 5,6-dihydro-1,4,2-dioxazin-3-ylpyridinesulfonyl iso(thio)cyanates of the formula (IV) is described further hereinbelow) with an aminoheterocycle of the formula (V)

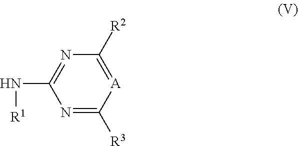

in which V, W, X, Y, R¹ to R⁸, Q, A and n have the above meanings. The corresponding aminopyrimidines and aminotriazines are commercially available.

In a third embodiment of the present invention, the compounds of the formula (I) are prepared by reacting sulfonyl (thio)carbamates of the formula (VI)

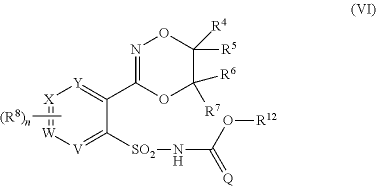

(the preparation of corresponding sulfonyl (thio)carbamates of the formula (VI) is described further hereinbelow), in which R¹² is a substituted or unsubstituted (C₁-C₂₀)-hydrocarbon radical such as aryl or alkyl, preferably optionally substituted phenyl or optionally substituted (C₁-C₄)-alkyl, with an amino heterocycle of the formula (V)

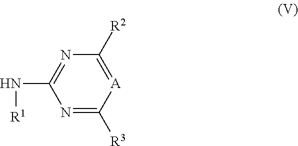

in which V, W, X, Y, R¹ to R⁸, Q, A and n have the abovementioned meanings.

In a fourth embodiment of the present invention, the compounds of the formula (I) are prepared by reacting (5,6-dihydro-1,4,2-dioxazin-3-yl)pyridylsulfonamides of the formula (II)

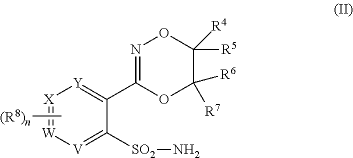

(the preparation of corresponding (5,6-dihydro-1,4,2-dioxazin-3-yl)pyridylsulfonamides of the formula (II) is described hereinabove) with an iso(thio)cyanate of the formula (VII)

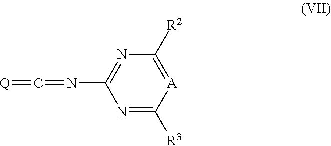

in which R¹ is hydrogen and R² to R⁸, V, W, X, Y, Q, n and A have the abovementioned meanings, optionally in the presence of a reaction auxiliary. Compounds of the formula (VII) are prepared by known methods from compounds of the formula (V) (R¹=hydrogen) in an inert solvent such as toluene or ethyl acetate and oxalyl chloride or phosgene (cf. preparation procedures in EP 0 388 994 A and EP 0 628 031 A).

In a fifth embodiment of the present invention, the compounds of the formula (I) are prepared by initially reacting, with base catalysis, an amino heterocycle of the formula (V)

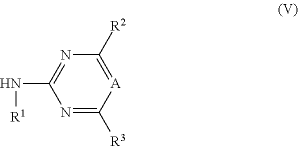

with a carbonic ester, for example diphenyl carbonate, and reacting, in a one-pot reaction, the resulting intermediate of the formula (III)

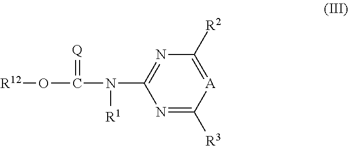

with a (5,6-dihydro-1,4,2-dioxazin-3-yl)pyridylsulfonamide of the formula (II)

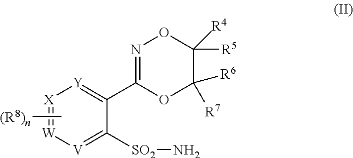

(cf. JP1989221366), in which V, W, X, Y, R¹ to R⁸, Q, A and n have the abovementioned meanings.

In a sixth embodiment of the present invention, the compounds of the formula (I) are prepared by reacting (5,6-dihydro-1,4,2-dioxazin-3-yl)pyridylsulfonyl halids of the formula (VIII)

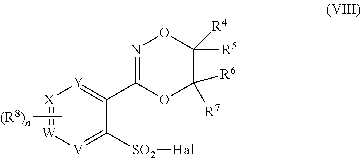

in which Hal is a halogen atom, preferably chlorine, with a (thio)cyanate, in particular a metal (thio)cyanate, in particular an alkali metal (thio)cyanate, such as sodium (thio)cyanate, to give an iso(thio)cyanate of the formula (IV)

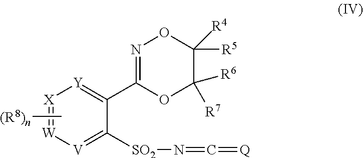

or a solvated (stabilized) derivative thereof, and subsequently with an amino heterocycle of the formula (V)

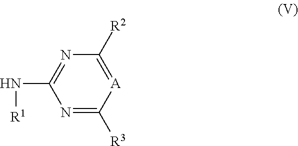

in which V, W, X, Y, R¹ to R⁸, Q, A and n have the abovementioned meanings.

In a seventh embodiment of the present invention, the compounds of the formula (I) where Q=oxygen are prepared by reacting (5,6-dihydro-1,4,2-dioxazin-3-yl)pyridylsulfonamides of the formula (II)

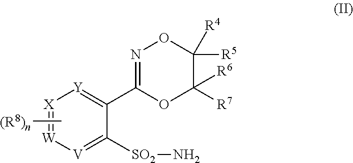

with a heterocyclic biscarbamate of the formula (IX),

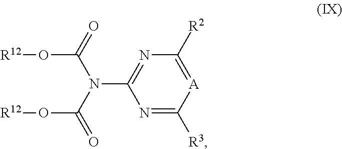

in which R¹² is a substituted or unsubstituted (C₁-C₂₀)-hydrocarbon radical such as aryl or alkyl, preferably optionally substituted phenyl or optionally substituted (C₁-C₄)-alkyl, in the presence of a basic reaction auxiliary, where V, W, X, Y, R¹ to R⁸, A and n have the abovementioned meanings. The compounds of the formula (IX) are known and can be prepared by known processes, cf. WO 96/022284 A.

In an eighth embodiment of the present invention, the compounds of the formula (I) are prepared by initially reacting, with base catalysis (5,6-dihydro-1,4,2-dioxazin-3-yl)pyridylsulfonamides of the formula (II)

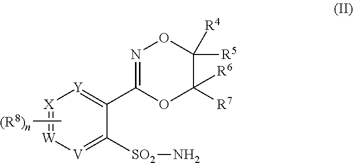

with a carbonic ester, for example diphenyl carbonate, and reacting, in a one-pot reaction, the resulting intermediate of the formula (VI)

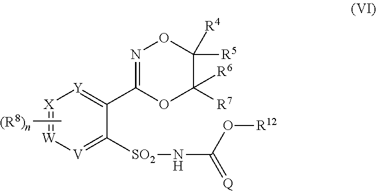

with an amino heterocycle of the formula (V)

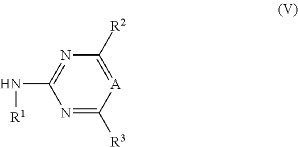

in which V, W, X, Y, R¹ to R⁸, Q, A and n have the abovementioned meanings.

All these processes lead to compounds of the formula (I) according to the invention.

Inert solvents are used in each case in each of the abovementioned process variants. For the purposes of the present invention, inert solvents are understood as meaning those which are inert under the respective reaction conditions, i.e. which, in particular, do not react with the starting materials, but which need not be inert under all reaction conditions.

Examples of organic solvents which can be used within the scope of the present invention are aromatic or aliphatic solvents such as benzene, toluene, xylene, mesitylene, hexane, heptane, octane, cyclohexane; aliphatic and aromatic halohydrocarbons such as methylene chloride, dichloroethane, chloroform, carbon tetrachloride, chlorobenzene, dichlorobenzene, ethers such as diethyl ether, dibutyl ether, diisobutyl ether, methyl tert-butyl ether, isopropyl ethyl ether, diisopropyl ether, tetrahydrofuran, and dioxane; furthermore also dimethyl sulfoxide and acid amide derivatives, such as N,N-dimethylformamide, N,N-dimethylacetamide and N-methyl-2-pyrrolidone, and also carboxylic acid esters such as ethyl acetate, or else diglymes, dimethyl glycol; nitriles such as acetonitrile, propionitrile or butyronitrile, and also ketones such as acetone, methyl ethyl ketone or cyclohexanone. Especially preferred are toluene, xylene, dichlorobenzene, chlorobenzene, acetonitrile, acetone, butyronitrile or ethyl acetate. However, the present invention is not limited to the solvents mentioned above by way of example.

The reaction temperature at which the reactions according to the above embodiments can be carried out may vary within wide ranges. For example, the reactions can be carried out at a temperature of from 0 to 100° C., preferably from 20 to 70° C.

In general, the reactions of the present inventions are carried out under atmospheric pressure. However, they may also be carried out under elevated pressure or reduced pressure—in general between 0.1 bar and 10 bar.

The processes for the preparation of the N-azinyl-N′-pyridylsulfonylureas of the formula (I) according to the invention are, where necessary, carried out in the presence of a basic reaction auxiliary.

Suitable reaction auxiliaries are all customary inorganic or organic bases. These include, for example, the hydrides, hydroxides, amides, alkoxides, acetates, carbonates or hydrogencarbonates of alkali metals or alkaline earth metals, such as, for example, lithium hydride, sodium hydride, potassium hydride, calcium hydride, lithium amide, sodium amide, potassium amide, sodium methoxide, potassium methoxide, sodium ethoxide, potassium ethoxide, sodium propoxide, potassium propoxide, aluminum isopropoxide, sodium tert-butoxide, potassium tert-butoxide, sodium hydroxide, potassium hydroxide, ammonium hydroxide, sodium acetate, potassium acetate, calcium acetate, ammonium acetate, sodium carbonate, potassium carbonate, calcium carbonate, ammonium carbonate, sodium hydrogencarbonate, potassium hydrogencarbonate, and basic organic nitrogen compounds such as trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl- and 4-methylpyridine, 2,4-dimethyl-, 2,6-dimethyl-, 3,4-dimethyl- and 3,5-dimethylpyridine, 5-ethyl-2-methylpyridine, N-methylpyridine, 4-(N,N-dimethylamino)pyridine, diazabicyclooctane (DABCO), diazabicyclononene (DBN) or diazabicycloundecene (DBU).

### Intermediates

Also subject matter of the present application are certain intermediates which are generated according to the above-shown synthetic routes when preparing the compounds of the formula (I) according to the invention.

The present intermediates initially show no preference for W, X or Y equals nitrogen. In principle, therefore, the following intermediates comprise in principle all compounds in which W equals nitrogen, X equals nitrogen or Y equals nitrogen. It is especially preferred when W or Y equal nitrogen. Even further preferred within the scope of the present invention is when W equals nitrogen in the intermediates which are described hereinbelow in greater detail. These intermediates lead to the synthesis of the preferred compounds of the formula (Ia).

In a first embodiment of the intermediates, accordingly, the present invention also comprises compounds of the formula (II)

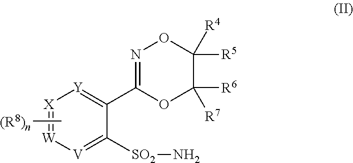

in which the radicals V, W, X, Y, R⁴, R⁵, R⁶, R⁷, R⁸ and n have the general, preferred and especially preferred meanings already indicated further hereinabove.

In a second embodiment of the intermediates, further subject matter of the present invention are also compounds of the formula (IV)

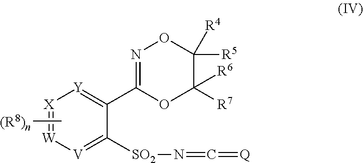

in which the radicals V, W, X, Y, R⁴, R⁵, R⁶, R⁷, R⁸, Q and n have the general, preferred and especially preferred meanings already indicated further hereinabove.

In a third embodiment of the intermediates, further subject matter of the present invention are also compounds of the formula (X)

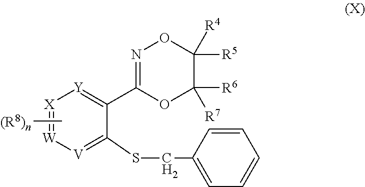

in which the radicals V, W, X, Y, R⁴, R⁵, R⁶, R⁷, R⁸ and n have the general, preferred and especially preferred meanings already indicated further hereinabove.

In a fourth embodiment of the intermediates, further subject matter of the present invention are also compounds of the formula (VIII)

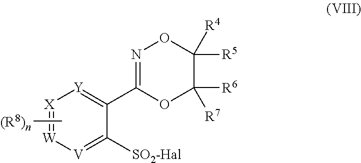

in which the radicals V, W, X, Y, R⁴, R⁵, R⁶, R⁷, R⁸, Hal and n have the general, preferred and especially preferred meanings already indicated further hereinabove.

In a fifth embodiment of the intermediates, further subject matter of the present invention are also compounds of the formula (VI)

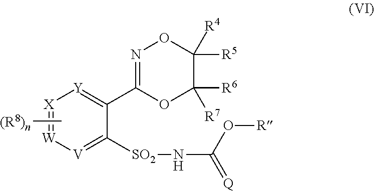

in which the radicals V, W, X, Y, R⁴, R⁵, R⁶, R⁷, R⁸, R″, Q and n have the general, preferred and especially preferred meanings already indicated further hereinabove.

Collections of compounds of the formula (I) and/or their salts which can be synthesized in accordance with the abovementioned reactions can also be prepared in a parallelized fashion, which can be carried out manually or in a partially automated or fully automated fashion. Here, it is possible for example to automate the procedure of the reaction, the work-up or the purification of the products or intermediates. Overall, this is understood as meaning a procedure as is described, for example, by D. Tiebes in Combinatorial Chemistry—Synthesis, Analysis, Screening (Editor Gunther Jung), Verlag Wiley 1999, on pages 1 to 34.

To carry out the reaction and the work-up in a parallelized fashion, it is possible to use a series of apparatuses which are commercially available, for example Calpyso reaction blocks from Barnstead International, Dubuque, Iowa 52004-0797, USA or reaction stations from Radleys, Shirehill, Saffron Walden, Essex, CB 11 3AZ, UK, or MuItiPROBE Automated Workstations from Perkin Elmer, Waltham, Mass. 02451, USA. To carry out a parallelized purification of compounds of the formula (I) and their salts, or the intermediates which are generated during the preparation, chromatographic apparatuses are available, inter alia, for example those from ISCO, Inc., 4700 Superior Street, Lincoln, Nebr. 68504, USA.

The apparatuses which have been listed lead to a modular procedure in which the individual passes are automated, but in which manual operations have to be carried out between the passes. This can be circumvented by employing partially or fully integrated automation systems where the respective automation modules are operated by, for example, robots. Said automation systems can be obtained commercially for example from Caliper, Hopkinton, Mass. 01748, USA.

Carrying out individual or a plurality of synthesis steps can be supported by using polymer-supported reagents/scavanger resins. A series of experimental protocols are described in the specialist literature, for example in ChemFiles, Vol. 4, No. 1, Polymer-Supported Scavengers and Reagents for Solution-Phase Synthesis (Sigma-Aldrich).

Compounds of the formula (I) and their salts can be prepared not only as in the methods described herein, but also fully or partially by solid-phase-supported methods. For this purpose, individual intermediates or all intermediates of the synthesis or of a synthesis adapted to suit the respective procedure are bound to a synthetic resin. Solid-phase-supported synthetic methods are described widely in the specialist literature, for example Barry A. Bunin in “The Combinatorial Index”, Academic Press, 1998 and Combinatorial Chemistry—Synthesis, Analysis, Screening (Editor Gunther Jung), Verlag Wiley, 1999. The use of solid-phase-supported synthetic methods permits a series of protocols known from the literature, which, in turn, can be carried out manually or in an automated fashion. For example, the reactions can be carried out by means of IRORI technology in microreactors from Nexus Biosystems, 12140 Community Road, Poway, Calif. 92064, USA.

Both in the solid phase and in the liquid phase, carrying out individual, or a plurality of, synthesis steps can be supported by using microwave technology. A series of experimental protocols are described in the specialist literature, for example in Microwaves in Organic and Medicinal Chemistry (Editors C. O. Kappe and A. Stadler), published by Wiley, 2005.

The preparation according to the processes described herein yields compounds of the formula (I) and their salts in the form of substance collections, which are referred to as libraries. Subject matter of the present invention are also libraries which comprise at least two compounds of the formula (I) and their salts.

Another subject matter of the invention owing to the herbicidal property of the compounds of the formula (I) is also the use of the compounds of the formula (I) according to the invention as herbicides for controlling harmful plants.

The application rate required of the compounds of the formula (I) varies as a function of the external conditions such as, inter alia, temperature, humidity and the nature of the herbicide used. It may vary within wide ranges, for example between 0.001 and 10 000 g/ha or more of active substance; preferably, however, it is between 0.5 and 5000 g/ha, by preference between 0.5 and 1000 g/ha and very especially preferably between 0.5 and 500 g/ha.

The compounds of the formula (I) (and/or their salts) according to the invention, hereinbelow referred to as “compounds according to the invention”, have an outstanding herbicidal activity against a broad spectrum of economically important monocotyledonous and dicotyledonous harmful annual plants. The active substances also have a good effect on perennial harmful plants which produce shoots from rhizomes, root stocks or other perennial organs and which are difficult to control.

Subject matter of the present invention is, therefore, also a method of controlling undesired plants or for regulating the growth of plants, preferably in crops of plants, wherein one or more compound(s) according to the invention is/are applied to the plants, (for example harmful plants such as monocotyledonous or dicotyledonous weeds or undesirable crop plants), to the seed (for example kernels, seeds or vegetative propagation organs such as tubers or shoot segments with buds) or the area on which the plants grow (for example the area under cultivation). In this context, the compounds according to the invention can be applied, for example, by the presowing method (if appropriate also by incorporation into the soil), pre-emergence or post-emergence method. By way of example, some represtnatives of the monocotyledonous and dicotyledonous weed flora which can be controlled by the compounds according to the invention may be mentioned individually, without the enumeration being intended as a limitation to certain species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

If the compounds according to the invention are applied to the surface before germination, either the emergence of the weed seedlings is prevented completely, or the weeds grow until they have reached the cotyledon stage, but then their growth is arrested and, ultimately, they die completely after three to four weeks have elapsed.

When, in the post-emergence method, the active substances are applied to the green plant parts, the growth is arrested after the treatment, and the harmful plants remain at the growth stage prevailing at the point in time of application or else die completely after a certain period of time has elapsed, so that, in this manner, competition by weeds, which is harmful to the crop plants, is eliminated at an early point in time and in a sustainable manner.

Although the compounds according to the invention have an outstanding herbicidal activity against monocotyledonous and dicotyledonous weeds, crop plants of economically important crops, for example of dicotyledonous crops of the genera Arachis, Beta, Brassica, Cucumis, Cucurbita, Helianthus, Daucus, Glycine, Gossypium, Ipomoea, Lactuca, Linum, Lycopersicon, Nicotiana, Phaseolus, Pisum, Solanum, Vicia, or of monocotyledonous crops of the genera Allium, Ananas, Asparagus, Avena, Hordeum, Oryza, Panicum, Saccharum, Secale, Sorghum, Triticale, Triticum, Zea, in particular Zea and Triticum, are damaged to a negligle extent only, or not at all, depending on the structure of the respective compound according to the invention and its application rate. The present compounds are therefore highly suitable for the selective control of undesired plant growth in plant crops such as stands of agriculturally useful plants or of ornamentals.

Moreover, the compounds according to the invention (depending on their respective structure and the application rate applied) have outstanding growth-regulatory properties in crop plants. They engage in the plants' metabolism in the regulating fashion and can therefore be employed for selectively affecting plant constituents and for facilitating harvesting, such as, for example, by triggering desiccation and stunted growth. Moreover, they are also suitable for the general control and inhibition of undesired vegetative growth without destroying the plants in the process. The inhibition of vegetative growth plays an important role in many monocotyledonous and dicotyledonous crops since for example lodging can thereby be reduced or prevented completely.

Due to the herbicidal and plant-growth-regulatory properties, the active substances can also be employed for controlling harmful plants in crops of plants which have been modified by genetic engineering or by traditional mutagenesis. As a rule, the transgenic plants are distinguished by particularly advantageous properties, for example by resistances to certain pesticides, mainly certain herbicides, resistances to plant diseases or to causative organisms of plant diseases such as certain insects or microorganisms such as fungi, bacteria or viruses. Other particular properties relate for example to the harvested crop in respect of quantity, quality, solubility, composition and specific constituents. Thus, transgenic plants with an increased starch content or a modified starch quality or those with a different fatty acid composition of the harvested crop are known.

Preferred with regard to transgenic crops is the use of the compounds according to the invention in economically important transgenic crops of useful plants and ornamentals, for example of cereals such as wheat, barley, rye, oats, millet/sorghum, rice and maize, or else crops of sugar beet, cotton, soya, oilseed rape, potato, tomato, pea and other vegetables.

Preferably, the compounds according to the invention can be employed as herbicides in crops of useful plants which are resistant, or have been made resistant by recombinant means, to the phytotoxic effects of the herbicides.

Traditional ways for generating novel plants which, in comparison with existing plants, have modified properties consist for example in classical breeding methods and the generation of mutants. Alternatively, it is possible to generate novel plants with modified properties with the aid of recombinant methods (see, for example, EP-A-0221044, EP-A-0131624). For example, the following have been described in several cases:
recombinant modifications of crop plants for modifying the starch which is synthesized in the plants (for example WO 92/11376, WO 92/14827, WO 91/19806),
transgenic crop plants which are resistant to certain herbicides of the glufosinate type (cf., for example, EP-A-0242236, EP-A-242246) or of the glyphosate type (WO 92/00377) or of the sulfonylurea type (EP-A-0257993, U.S. Pat. No. 5,013,659),
transgenic crop plants, for example cotton, with the ability of producing Bacillus thuringiensis toxins (Bt toxins), which make the plants resistant to certain pests (EP-A-0142924, EP-A-0193259),
transgenic crop plants with a modified fatty acid composition (WO 91/13972),
recombinantly modified crop plants with novel constituents or secondary metabolites, for example novel phytoalexins, which bring about increased disease resistance (EPA 309862, EPA0464461),
recombinantly modified plants with reduced photorespiration which feature higher yields and higher stress tolerance (EPA 0305398),
transgenic crop plants which produce pharmaceutically or diagnostically important proteins (“molecular pharming”),
transgenic crop plants which are distinguished by higher yields or better quality,
transgenic crop plants which are distinguished by a combination for example of the abovementioned novel properties (“gene stacking”).

A large number of molecular-biological techniques with the aid of which novel transgenic plants with modified properties can be generated are known in principle, see, for example, I. Potrykus and G. Spangenberg (eds.) Gene Transfer to Plants, Springer Lab Manual (1995), Springer Verlag Berlin, Heidelberg or Christou, “Trends in Plant Science” 1 (1996) 423-431).

To carry out such recombinant manipulations, it is possible to introduce, into plasmids, nucleic acid molecules which permit a mutagenesis or a sequence modification by recombining DNA sequences. With the aid of standard methods, for example, it is possible to carry out base substitutions, to remove part-sequences or to add natural or synthetic sequences. To link the DNA fragments to each other, it is possible to add adapters or linkers to the fragments, see, for example, Sambrook et al., 1989, Molecular Cloning, A Laboratory Manual, 2nd Ed., Cold Spring Harbor Laboratory Press, Cold Spring Harbor, N.Y.; or Winnacker “Gene and Klone” [genes and clones], VCH Weinheim 2nd Ed. 1996.

The generation of plant cells with a reduced activity of a gene product can be achieved for example by expression of at least one corresponding antisense RNA, a sense RNA for obtaining a cosuppression effect or the expression of at least one suitably constructed ribozyme which specifically cleaves transcripts of the abovementioned gene product.

Here, it is possible firstly to use DNA molecules which comprise the entire coding sequence of a gene product including any flanking sequences which may be present, or else DNA molecules which only comprise parts of the coding sequence, but these parts must be sufficiently long for bringing about an antisense effect in the cells. Another possibility is the use of DNA sequences which have a high degree of homology to the coding sequences of a gene product, but are not entirely identical.

When expressing nucleic acid molecules in plants, the protein synthetized can be localized in any compartment of the plant cell. To achieve localization in a particular compartment, however, it is possible for example to link the coding region to DNA sequences which ensure the localization in a particular compartment. Such sequences are known to the skilled worker (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). Expression of the nucleic acid molecules may also take place in the organelles of the plant cells.

The transgenic plant cells can be regenerated by known techniques to give intact plants. The transgenic plants may, in principle, take the form of plants of any plant species, i.e. both monocotyledonous and dicotyledonous plants.

Thus, it is possible to obtain transgenic plants which feature modified characteristics due to overexpression, suppression or inhibition of homologous (=natural) genes or gene sequences or by expressing heterologous (=foreign) genes or gene sequences.

The compounds (I) according to the invention can preferably be employed in transgenic crops which are resistant to growth substances, such as, for example, dicamba, or against herbicides which inhibit essential plant enzymes, for example acetolactate synthases (ALS), EPSP synthases, glutamine synthases (GS) or hydroxyphenylpyruvate dioxygenases (HPPD), or against herbicides from the group of the sulfonylureas, glyphosate, glufosinate or benzoylisoxazoles and analogous active substances, respectively.

When the active substances according to the invention are employed in transgenic crops, they show effects against harmful plants which can also be observed in other crops, but frequently also effects which are specific to the application in the respective transgenic crop, for example a modified or specifically widened weed spectrum which can be controlled, modified application rates which can be employed, preferably good combining ability with the herbicides to which the transgenic crop is resistant, and an effect on growth and yield of the transgenic crop plants.

The invention therefore also relates to the use of the compounds of the formula (I) according to the invention as herbicides for controlling harmful plants in transgenic crop plants.

The compounds according to the invention can be employed in the customary preparations in the form of wettable powders, emulsifiable concentrates, sprayable solutions, dusts or granules. The invention therefore also relates to herbicidal and plant-growth-regulatory compositions which comprise the compounds according to the invention.

The compounds according to the invention can be formulated in various ways, depending on the prevailing biological and/or chemical-physical parameters. The following are examples of possible formulations: wettable powders (WP), water-soluble powders (SP), water-soluble concentrates, emulsifiable concentrates (EC), emulsions (EW) such as oil-in-water and water-in-oil emulsions, sprayable solutions, suspension concentrates (SC), oil- or water-based dispersions, oil-miscible solutions, capsule suspensions (CS), dusts (DP), seed-dressing products, granules for broadcasting and soil application, granules (GR) in the form of microgranules, spray granules, absorption granules and adsorption granules, water-dispersible granules (WG), water-soluble granules (SG), ULV formulations, microcapsules and waxes. These individual formulation types are known in principle and are described, for example, in: Winnacker-Kuchler, “Chemische Technologie” [chemical technology], Volume 7, C. Hauser Verlag Munich, 4th Edition 1986; Wade van Valkenburg, “Pesticide Formulations”, Marcel Dekker, N.Y., 1973; K. Martens, “Spray Drying” Handbook, 3rd Ed. 1979, G. Goodwin Ltd. London.

The formulation auxiliaries required, such as inert materials, surfactants, solvents and further additives are likewise known and are described, for example, in: Watkins, “Handbook of Insecticide Dust Diluents and Carriers”, 2nd Ed., Darland Books, Caldwell N.J., H.v. Olphen, “Introduction to Clay Colloid Chemistry”; 2nd Ed., J. Wiley & Sons, N.Y.; C. Marsden, “Solvents Guide”; 2nd Ed., Interscience, N.Y. 1963; McCutcheon's “Detergents and Emulsifiers. Annual”, MC Publ. Corp., Ridgewood N.J.; Sisley and Wood, “Encyclopedia of Surface Active Agents”, Chem. Publ. Co. Inc., N.Y. 1964; Schönfeldt, “Grenzflächenaktive Äthylenoxidaddukte” [interface-active ethylene oxide adducts], Wiss. Verlagsgesell., Stuttgart 1976; Winnacker-Küchler, “Chemische Technologie”, Volume 7, C. Hauser Verlag Munich, 4th Edition 1986.

Based on these formulations, it is also possible to prepare combinations with other pesticidally active substances such as, for example, insecticides, akaricides, herbicides, fungicides, and also with safeners, fertilizers and/or growth regulators, for example in the form of a ready mix or a tank mix.

Wettable powders are preparations which are uniformly dispersible in water and which, besides a diluent or inert substance, also comprise ionic and/or nonionic surfactants (wetting agents, dispersants) in addition to the active substance, for example polyoxyethylated alkylphenols, polyoxethylated fatty alcohols, polyoxethylated fatty amines, fatty alcohol polyglycol ether sulfates, alkanesulfonates, alkylbenzenesulfonates, sodium ligninsulfonate, sodium 2,2′-dinaphthylmethane-6,6′-disulfonate, sodium dibutylnaphthalenesulfonate or else sodium oleoylmethyltaurite. To prepare the wettable powders, the herbicidal active substances are ground finely, for example in customary apparatuses such as hammer mills, blower mills and air-jet mills, and simultaneously or subsequently mixed with the formulation auxiliaries.

Emulsifiable concentrates are prepared by dissolving the active substance in an organic solvent, for example butanol, cyclohexanone, dimethylformamide, xylene or else higher-boiling aromatics or hydrocarbons or mixtures of the organic solvents, with addition of one or more ionic and/or nonionic surfactants (emulsifiers). Examples of emulsifiers which can be used are: calcium salts of alkylarylsulfonic acids, such as calcium dodecylbenzenesulfonate, or nonionic emulsifiers such as fatty acid polyglycol esters, alkylarylpolyglycol ethers, fatty alcohol polyglycol ethers, propylene oxide/ethylene oxide condensates, alkyl polyethers, sorbitan esters such as, for example, sorbitan fatty acid esters or polyoxyethylene sorbitan esters such as, for example, polyoxyethylene sorbitan fatty acid esters.

Dusts are obtained by grinding the active substance with finely divided solid materials, for example talc, natural clays such as kaolin, bentonite and pyrophyllite, or diatomaceous earth.

Suspension concentrates may be water- or oil-based. They can be prepared for example by wet-grinding using commercially available bead mills and, if appropriate, an addition of surfactants as they have already been listed for example above in the case of the other formulation types.

Emulsions, for example oil-in-water emulsions (EW), can be prepared for example by means of stirrers, colloid mills and/or static mixers using aqueous-organic solvents and, if appropriate, surfactants as they have already been listed for example above in the case of the other formulation types.

Granules can be prepared either by spraying the active substance onto adsorptive granulated inert material or by applying active substance concentrates to the surface of carriers such as sand, kaolinites or granulated inert material by means of binders, for example polyvinyl alcohol, sodium polyacrylate or else mineral oils. Suitable active substances can also be granulated in the manner which is conventionally used for the preparation of fertilizer granules, if appropriate as a mixture with fertilizers.

As a rule, water-dispersible granules are prepared by the customary methods such as spray-drying, fluidized-bed granulation, disk granulation, mixing in high-speed mixers and extrusion without solid inert material.

To prepare disk, fluidized-bed, extruder and spray granules, see, for example, the methods in “Spray-Drying Handbook” 3rd Ed. 1979, G. Goodwin Ltd., London; J. E. Browning, “Agglomeration”, Chemical and Engineering 1967, pages 147 et seq.; “Perry's Chemical Engineer's Handbook”, 5th Ed., McGraw-Hill, New York 1973, p. 8-57.

For further details on the formulation of plant protection products, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

As a rule, the agrochemical preparations comprise from 0.1 to 99% by weight, in particular from 0.1 to 95% by weight, of compounds according to the invention. In wettable powders, the active substance concentration amounts to for example approximately 10 to 90% by weight, the remainder to 100% is composed of conventional formulation components. In the case of emulsifiable concentrates, the active substance concentration may be approximately 1 to 90, preferably from 5 to 80% by weight. Formulations in the form of dust comprise from 1 to 30% by weight of active substance, preferably in most cases from 5 to 20% by weight of active substance, while sprayable solutions comprise from approximately 0.05 to 80, preferably from 2 to 50% by weight of active substance. In the case of water-dispersible granules, the active substance content depends partly on whether the active compound is present in liquid or solid form and on the granulation auxiliaries, fillers and the like which are used. In the case of the water-dispersible granules, the active substance content is, for example, between 1 and 95% by weight, preferably between 10 and 80% by weight.

In addition, the abovementioned active substance formulation comprise, if appropriate, the adhesives, wetters, dispersants, emulsifiers, penetrants, preservatives, antifreeze agents, solvents, fillers, carriers, colorants, antifoam agents, evaporation inhibitors and agents which affect the pH and the viscosity which are customary in each case.

The compounds of the formula (I) or their salts can be employed as such or in the form of their preparations (formulations) as a combination with other pesticidally active substances such as, for example, insecticides, acaricides, nematicides, herbicides, fungicides, safeners, fertilizers and/or growth regulators, for example as a ready mix or as tank mixes.

Combination partners which can be used for the compounds according to the invention in mixed formulations or in a tank mix are, for example, known active substances which are based on the inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II, protoporphyrinogen oxidase, as they are described in, for example, Weed Research 26 (1986) 441-445 or “The Pesticide Manual”, 13th edition, The British Crop Protection Council and the Royal Soc. of Chemistry, 2003 and the literature cited therein. Known herbicides or plant growth regulators which can be combined with the compounds according to the invention and which should be mentioned are, for example, the following active substances (the compounds are either given by their “common name” according to the International Organization for Standardization (ISO) or by their chemical name or the code number) and always comprise all use forms such as acids, salts, esters and isomers and also stereoisomers and optical isomers. In this context, one and in some cases also a plurality of use forms are mentioned by way of example:

Acetochlor, acibenzolar, acibenzolar-S-methyl, acifluorfen, acifluorfen-sodium, aclonifen, alachlor, allidochlor, alloxydim, alloxydim-sodium, ametryn, amicarbazone, amidochlor, amidosulfuron, aminocyclopyrachlor, aminopyralid, amitrole, ammonium sulfamate, ancymidol, anilofos, asulam, atrazine, azafenidin, azimsulfuron, aziprotryn, BAH-043, BAS-140H, BAS-693H, BAS-714H, BAS-762H, BAS-776H, BAS-800H, beflubutamid, benazolin, benazolin-ethyl, bencarbazone, benfluralin, benfuresate, bensulide, bensulfuron-methyl, bentazone, benzfendizone, benzobicyclon, benzofenap, benzofluor, benzoylprop, bifenox, bilanafos, bilanafos-sodium, bispyribac, bispyribac sodium, bromacil, bromobutide, bromofenoxim, bromoxynil, bromuron, buminafos, busoxinone, butachlor, butafenacil, butamifos, butenachlor, butralin, butroxydim, butylate, cafenstrole, carbetamide, carfentrazone, carfentrazone-ethyl, chlomethoxyfen, chloramben, chlorazifop, chlorazifop-butyl, chlorbromuron, chlorbufam, chlorfenac, chlorfenac-sodium, chlorfenprop, chlorflurenol, chlorflurenol-methyl, chloridazon, chlorimuron, chlorimuron-ethyl, chlormequat chloride, chlornitrofen, chlorophthalim, chlorthal-dimethyl, chlorotoluron, chlorsulfuron, cinidon, cinidon-ethyl, cinmethylin, cinosulfuron, clethodim, clodinafop clodinafop-propargyl, clofencet, clomazone, clomeprop, cloprop, clopyralid, cloransulam, cloransulam-methyl, cumyluron, cyanamide, cyanazine, cyclanilide, cycloate, cyclosulfamuron, cycloxydim, cycluron, cyhalofop, cyhalofop-butyl, cyperquat, cyprazine, cyprazole, 2,4-D, 2,4-DB, daimuron/dymrone, dalapon, daminozide, dazomet, n-decanol, desmedipham, desmetryn, detosyl pyrazolate (DTP), di-allate, dicamba, dichlobenil, dichlorprop, dichlorprop-P, diclofop, diclofop-methyl, diclofop-P-methyl, diclosulam, diethatyl, diethatyl-ethyl, difenoxuron, difenzoquat, diflufenican, diflufenzopyr, diflufenzopyr-sodium, dimefuron, dikegulac-sodium, dimefuron, dimepiperate, dimethachlor, dimethametryn, dimethenamid, dimethenamid-P, dimethipin, dimetrasulfuron, dinitramine, dinoseb, dinoterb, diphenamid, dipropetryn, diquat, diquat dibromide, dithiopyr, diuron, DNOC, eglinazine-ethyl, endothal, EPTC, esprocarb, ethalfiuralin, ethametsulfuron-methyl, ethephon, ethidimuron, ethiozin, ethofumesate, ethoxyfen, ethoxyfen-ethyl, ethoxysulfuron, etobenzanid, F-5331, i.e. N-[2-chloro-4-fluoro-5-[4-(3-fluoropropyl)-4,5-dihydro-5-oxo-1H-tetrazol-1-yl]phenyl]ethanesulfonamide, fenoprop, fenoxaprop, fenoxaprop-P, fenoxaprop-ethyl, fenoxaprop-P-ethyl, fenoxasulfone, fentrazamide, fenuron, flamprop, flamprop-M-isopropyl, flamprop-M-methyl, flazasulfuron, florasulam, fluazifop, fluazifop-P, fluazifop-butyl, fluazifop-P-butyl, fluazolate, flucarbazone, flucarbazone-sodium, flucetosulfuron, fluchloralin, flufenacet (thiafluamide), flufenpyr, flufenpyr-ethyl, flumetralin, flumetsulam, flumiclorac, flumiclorac-pentyl, flumioxazin, flumipropyn, fluometuron, fluorodifen, fluoroglycofen, fluoroglycofen-ethyl, flupoxam, flupropacil, flupropanate, flupyrsulfuron, flupyrsulfuron-methyl sodium, flurenol, flurenol-butyl, fluridone, fluorochloridone, fluoroxypyr, fluoroxypyr-meptyl, flurprimidol, flurtamone, fluthiacet, fluthiacet-methyl, fluthiamide, fomesafen, foramsulfuron, forchlorfenuron, fosamine, furyloxyfen, gibberellic acid, glufosinate, L-glufosinate, L-glufosinate-ammonium, glufosinate-ammonium, glyphosate, glyphosate-isopropylammonium, H-9201, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HNPC-9908, HOK-201, HW-02, imazamethabenz, imazamethabenz-methyl, imazamox, imazapic, imazapyr, imazaquin, imazethapyr, imazosulfuron, inabenfide, indanofan, indaziflam, indoleacetic acid (IAA); 4-indol-3-ylbutyric acid (IBA), iodosulfuron, iodosulfuron-methyl sodium, ioxynil, ipfencarbazone, isocarbamid, isopropalin, isoproturon, isouron, isoxaben, isoxachlortole, isoxaflutole, isoxapyrifop, IDH-100, KUH-043, KUH-071, karbutilate, ketospiradox, lactofen, lenacil, linuron, maleic hydrazide, MCPA, MCPB, MCPB-methyl, -ethyl and -sodium, mecoprop, mecoprop-sodium, mecoprop-butotyl, mecoprop-P-butotyl, mecoprop-P-dimethylammonium, mecoprop-P-2-ethylhexyl, mecoprop-P-potassium, mefenacet, mefluidide, mepiquat chloride, mesosulfuron, mesosulfuron-methyl, mesotrione, methabenzthiazuron, metam, metamifop, metamitron, metazachlor, methazole, methiozolin, methoxyphenone, methyldymron, 1-methylcyclopropene, methyl isothiocyanate, metobenzuron, metobromuron, metolachlor, S-metolachlor, metosulam, metoxuron, metribuzin, metsulfuron, metsulfuron-methyl, molinate, monalide, monocarbamide, monocarbamide dihydrogen sulfate, monolinuron, monosulfuron, monuron, MT 128, MT-5950, i.e. N-[3-chloro-4-(1-methylethyl)-phenyl]-2-methylpentanamide, NGGC-011, naproanilide, napropamide, naptalam, NC-310, i.e. 4-(2,4-dichlorobenzoyl)-1-methyl-5-benzyloxypyrazole, neburon, nicosulfuron, nipyraclofen, nitralin, nitrofen, nitrophenolate-sodium (isomer mixture), nitrofluorfen, nonanoic acid, norflurazon, orbencarb, orthosulfamuron, oryzalin, oxadiargyl, oxadiazon, oxasulfuron, oxaziclomefone, oxyfluorfen, paclobutrazole, paraquat, paraquat dichloride, pelargonic acid (nonanoic acid), pendimethalin, pendralin, penoxsulam, pentanochlor, pentoxazone, perfluidone, pethoxamid, phenisopham, phenmedipham, phenmedipham-ethyl, picloram, picolinafen, pinoxaden, piperophos, pirifenop, pirifenop-butyl, pretilachlor, primisulfuron, primisulfuron-methyl, probenazole, profluazol, procyazine, prodiamine, prifluraline, profoxydim, prohexadione, prohexadione-calcium, prohydrojasmone, prometon, prometryn, propachlor, propanil, propaquizafop, propazine, propham, propisochlor, propoxycarbazone, propoxycarbazone-sodium, propyrisulfuron, propyzamide, prosulfalin, prosulfocarb, prosulfuron, prynachlor, pyraclonil, pyraflufen, pyraflufen-ethyl, pyrasulfotole, pyrazolynate (pyrazolate), pyrazosulfuron-ethyl, pyrazoxyfen, pyribambenz, pyribambenz-isopropyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, secbumeton, sethoxydim, siduron, simazine, simetryn, SN-106279, sulcotrione, sulfallate (CDEC), sulfentrazone, sulfometuron, sulfometuron-methyl, sulfosate (glyphosate-trimesium), sulfosulfuron, SYN-523, SYP-249, SYP-298, SYP-300, tebutam, tebuthiuron, tecnazene, tefuryltrione, tembotrione, tepraloxydim, terbacil, terbucarb, terbuchlor, terbumeton, terbuthylazine, terbutryn, TH-547, thenylchlor, thiafluamide, thiazafluoron, thiazopyr, thidiazimin, thidiazuron, thiencarbazone, thiencarbazone-methyl, thifensulfuron, thifensulfuron-methyl, thiobencarb, tiocarbazil, topramezone, tralkoxydim, tri-allate, triasulfuron, triaziflam, triazofenamide, tribenuron, tribenuron-methyl, trichloroacetic acid (TCA), triclopyr, tridiphane, trietazine, trifloxysulfuron, trifloxysulfuron-sodium, trifluralin, triflusulfuron, triflusulfuron-methyl, trimeturon, trinexapac, trinexapac-ethyl, tritosulfuron, tsitodef, uniconazole, uniconazole-P, vernolate, ZJ-0166, ZJ-0270, ZJ-0543, ZJ-0862 and the following compounds:

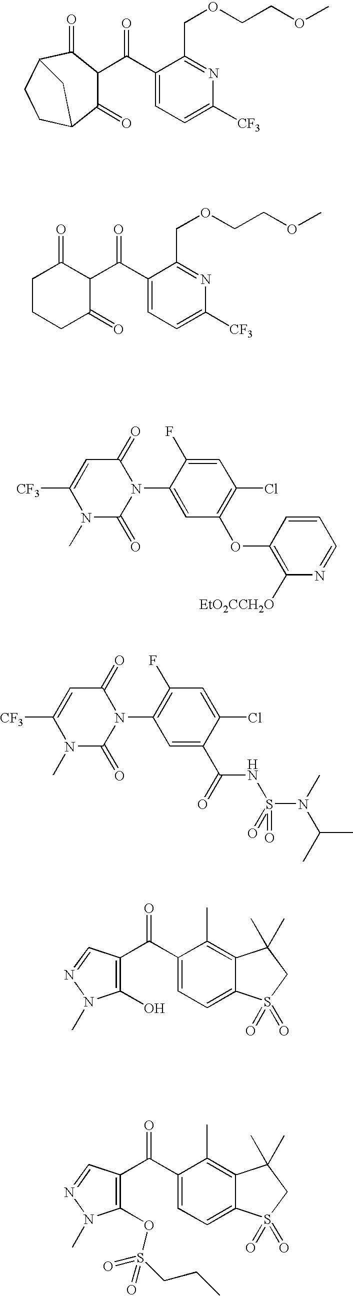

The selective control of harmful plants in crops of useful plants and ornamentals is of particular interest. Although the compounds of the formula (I) according to the invention already display very good to sufficient selectivity in many crops, phytotoxicity symptoms on the crop plants may occur, in principle, in some crops and especially also in the case of mixtures with other herbicides which are less selective. In this respect, combinations of compounds of the formula (I) according to the invention which are of particular interest are those which comprise the compounds of the formula (I) or their combinations with other herbicides or pesticides and safeners. The safeners, which are employed in an antidote-effective content, reduce the phytotoxic side-effects of the herbicides/pesticides employed, for example economically important crops such as cereals (wheat, barley, rye, maize, rice, millet/sorghum), sugar beet, sugar cane, oilseed rape, cotton and soya beans, preferably cereals. The following groups of compounds are examples which are suitable as safeners for the compounds of the formula (I) or their salts, alone or else in their combinations with further pesticides:

The safeners are preferably selected from the group consisting of:
S1) Compounds of the formula (S1)

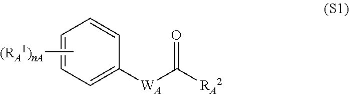

where the symbols and indices have the following meanings:
n_{A} is a natural number from 0 to 5, preferably from 0 to 3;
R_{A}¹ is halogen, (C₁-C₄)-alkyl, (C₁-C₄)-alkoxy, nitro or (C₁-C₄)-haloalkyl;
W_{A} is an unsubstituted or substituted divalent heterocyclic radical from the group consisting of partially unsaturated or aromatic five-membered heterocycles having 1 to 3 heteroring atoms of the group N and O, where at least one nitrogen atom and at most one oxygen atom is present in the ring, preferably a radical from the group consisting of (W_{A}¹) to (W_{A}⁴),

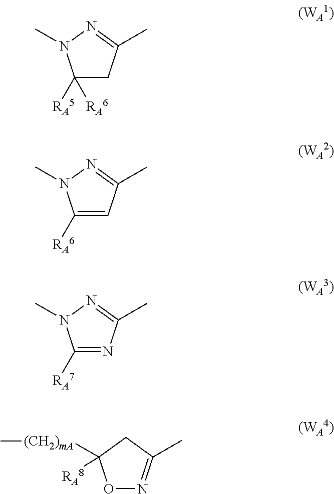

m_{A} is 0 or 1;
R_{A}² is OR_{A}³, SR_{A}³ or NR_{A}³R_{A}⁴ or a saturated or unsaturated 3- to 7-membered heterocycle having at least one nitrogen atom and up to 3 heteroatoms, preferably from the group consisting of O and S, which is attached via the nitrogen atom to the carbonyl group in (S1) and which is unsubstituted or substituted by radicals from the group consisting of (C₁-C₄)alkyl, (C₁-C₄)alkoxy and optionally substituted phenyl, preferably a radical of the formula OR_{A}³, NHR_{A}⁴ or N(CH₃)₂, in particular of the formula OR_{A}³;
R_{A}³ is hydrogen or an unsubstituted or substituted aliphatic hydrocarbon radical having preferably a total of 1 to 18 carbon atoms;
R_{A}⁴ is hydrogen, (C₁-C₆)alkyl, (C₁-C₆)alkoxy or substituted or unsubstituted phenyl;
R_{A}⁵ is H, (C₁-C₈)alkyl, (C₁-C₈)haloalkyl, (C₁-C₄)alkoxy-(C₁-C₈)alkyl, cyano or COOR_{A}⁹ where R_{A}⁹ is hydrogen, (C₁-C₈)alkyl, (C₁-C₈)haloalkyl, (C₁-C₄)alkoxy-(C₁-C₄)alkyl, (C₁-C₆)hydroxyalkyl, (C₃-C₁₂)cycloalkyl or tri-(C₁-C₄)alkylsilyl;
R_{A}⁶, R_{A}⁷, R_{A}⁸ are identical or different and are hydrogen, (C₁-C₈)alkyl, (C₁-C₈)haloalkyl, (C₃-C₁₂)cycloalkyl or substituted or unsubstituted phenyl;
   preferably:
a) compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds, as described in WO-A-91/07874;
b) derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4), and related compounds, as described in EP-A-333 131 and EP-A-269 806;
c) derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds, as described, for example, in EP-A-268554;
d) compounds of the triazolecarboxylic acid type, preferably compounds such as fenchlorazole(-ethyl), i.e. ethyl 1-(2,4-dichlorophenyl)-5-trichloro-methyl-(1H)-1,2,4-triazole-3-carboxylate (S1-7), and related compounds, as described in EP-A-174 562 and EP-A-346 620;
e) compounds of the 5-benzyl- or 5-phenyl-2-isoxazoline-3-carboxylic acid or the 5,5-diphenyl-2-isoxazoline-3-carboxylic acid (S1^{e}) type, preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds, as described in WO-A-91/08202, or 5,5-diphenyl-2-isoxazolinecarboxylic acid (S1-10) or ethyl 5,5-diphenyl-2-isoxazolinecarboxylate (S1-11) (“isoxadifen-ethyl”) or n-propyl 5,5-diphenyl-2-isoxazolinecarboxylate (S1-12) or ethyl 5-(4-fluorophenyl)-5-phenyl-2-isoxazoline-3-carboxylate (S1-13), as described in the patent application WO-A-95/07897.
S2) Quinoline derivatives of the formula (S2)

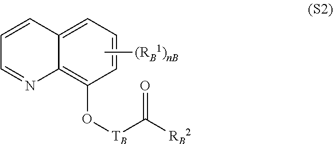

where the symbols and indices have the following meanings:
R_{B}¹ is halogen, (C₁-C₄)alkyl, (C₁-C₄)alkoxy, nitro or (C₁-C₄)haloalkyl;
n_{B} is a natural number from 0 to 5, preferably from 0 to 3;
R_{B}² is OR_{B}³, SR_{B}³ or NR_{B}³R_{B}⁴ or a saturated or unsaturated 3- to 7-membered heterocycle having at least one nitrogen atom and up to 3 heteroatoms, preferably from the group consisting of O and S, which is attached via the nitrogen atom to the carbonyl group in (S2) and is unsubstituted or substituted by radicals from the group consisting of (C₁-C₄)alkyl, (C₁-C₄)alkoxy or optionally substituted phenyl, preferably a radical of the formula OR_{B}³, NHR_{B}⁴ or N(CH₃)₂, in particular of the formula OR_{B}³;
R_{B}³ is hydrogen or an unsubstituted or substituted aliphatic hydrocarbon radical having preferably a total of 1 to 18 carbon atoms;
R_{B}⁴ is hydrogen, (C₁-C₆)alkyl, (C₁-C₆)alkoxy or substituted or unsubstituted phenyl;
T_{B} is a (C₁- or C₂)alkanediyl chain which is unsubstituted or substituted by one or two (C₁-C₄)alkyl radicals or by [(C₁-C₃)alkoxy]carbonyl;
   preferably:
a) compounds of the 8-quinolinoxyacetic acid (S2^{a}) type, preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5), methyl (5-chloro-8-quinolinoxy)acetate (S2-6), allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86 750, EP-A-94 349 and EP-A-191 736 or EP-A-0 492 366, and (5-chloro-8-quinolinoxy)acetic acid (S2-10), its hydrates and salts, for example its lithium, sodium, potassium, calcium, magnesium, aluminum, iron, ammonium, quarternary ammonium, sulfonium or phosphonium salts, as described in WO-A-2002/34048;
b) compounds of the (5-chloro-8-quinolinoxy)malonic acid (S2^{b}) type, preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Compounds of the formula (S3)

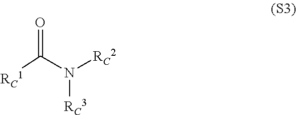

where the symbols and indices have the following meanings:
R_{C}¹ is (C₁-C₄)alkyl, (C₁-C₄)haloalkyl, (C₂-C₄)alkenyl, (C₂-C₄)haloalkenyl, (C₃-C₇)cycloalkyl, preferably dichloromethyl;
R_{C}², R_{C}³ are identical or different and are hydrogen, (C₁-C₄)alkyl, (C₂-C₄)alkenyl, (C₂-C₄)alkynyl, (C₁-C₄)haloalkyl, (C₂-C₄)haloalkenyl, (C₁-C₄)alkylcarbamoyl-(C₁-C₄)alkyl, (C₂-C₄)alkenylcarbamoyl-(C₁-C₄)alkyl, (C₁-C₄)alkoxy-(C₁-C₄)alkyl, dioxolanyl-(C₁-C₄)alkyl, thiazolyl, furyl, furylalkyl, thienyl, piperidyl, substituted or unsubstituted phenyl, or R_{C}² and R_{C}³ together form a substituted or unsubstituted heterocyclic ring, preferably an oxazolidine, thiazolidine, piperidine, morpholine, hexahydropyrimidine or benzoxazine ring;
   preferably
   active substances of the dichloroacetamide type which are frequently used as pre-emergence safeners (soil-acting safeners), such as, for example,
   “dichlormid” (=N,N-diallyl-2,2-dichloroacetamide) (S3-1),
   “R-29148” (=3-dichloroacetyl-2,2,5-trimethyl-1,3-oxazolidine) from Stauffer (S3-2),
   “R-28725” (=3-dichloroacetyl-2,2-dimethyl-1,3-oxazolidine) from Stauffer (S3-3),
   “benoxacor” (=4-dichloroacetyl-3,4-dihydro-3-methyl-2H-1,4-benzoxazine) (S3-4),
   “PPG-1292” (=N-allyl-N-[(1,3-dioxolan-2-yl)methyl]dichloroacetamide) from PPG Industries (S3-5),
   “DKA-24” (=N-allyl-N—[(allylaminocarbonyl)methyl]dichloroacetamide) from Sagro-Chem (S3-6),
   “AD-67” or “MON 4660” (=3-dichloroacetyl-1-oxa-3-azaspiro[4,5]decane) from Nitrokemia or Monsanto (S3-7),
   “TI-35” (=1-dichloroacetylazepane) from TRI-Chemical RT (S3-8),
   “diclonon” (dicyclonone) or “BAS145138” or “LAB145138” (S3-9) ((RS)-1-dichloroacetyl-3,3,8a-trimethylperhydropyrrolo[1,2-a]pyrimidin-6-one) from BASF,
   “furilazole” or “MON 13900” ((RS)-3-dichloroacetyl-5-(2-furyl)-2,2-dimethyl-oxazolidine) (S3-10) and its (R) isomer (S3-11).
S4) N-Acylsulfonamides of the formula (S4) and their salts

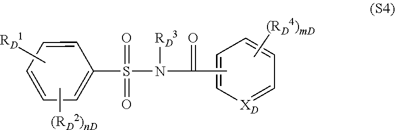

in which the symbols and indices have the following meanings
X_{D} is CH or N;
R_{D}¹ is CO—NR_{D}⁵R_{D}⁶ or NHCO—R_{D}⁷;
R_{D}² is halogen, (C₁-C₄)haloalkyl, (C₁-C₄)haloalkoxy, nitro, (C₁-C₄)alkyl, (C₁-C₄)alkoxy, (C₁-C₄)alkylsulfonyl, (C₁-C₄)alkoxycarbonyl or (C₁-C₄)alkylcarbonyl;
R_{D}³ is hydrogen, (C₁-C₄)alkyl, (C₂-C₄)alkenyl or (C₂-C₄)alkynyl;
R_{D}⁴ is halogen, nitro, (C₁-C₄)alkyl, (C₁-C₄)haloalkyl, (C₁-C₄)haloalkoxy, (C₃-C₆)cycloalkyl, phenyl, (C₁-C₄)alkoxy, cyano, (C₁-C₄)alkylthio, (C₁-C₄)alkylsulfinyl, (C₁-C₄)alkylsulfonyl, (C₁-C₄)alkoxycarbonyl or (C₁-C₄)alkylcarbonyl;
R_{D}⁵ is hydrogen, (C₁-C₆)alkyl, (C₃-C₆)cycloalkyl, (C₂-C₆)alkenyl, (C₂-C₆)alkynyl, (C₅-C₆)cycloalkenyl, phenyl or 3- to 6-membered heterocyclyl containing v_{D} heteroatoms from the group consisting of nitrogen, oxygen and sulfur, where the seven last mentioned radicals are substituted by v_{D} substituents from the group consisting of halogen, (C₁-C₆)alkoxy, (C₁-C₆)haloalkoxy, (C₁-C₂)alkylsulfinyl, (C₁-C₂)alkylsulfonyl, (C₃-C₆)cycloalkyl, (C₁-C₄)alkoxycarbonyl, (C₁-C₄)alkylcarbonyl and phenyl and, in the case of cyclic radicals, also (C₁-C₄)alkyl and (C₁-C₄)haloalkyl;
R_{D}⁶ is hydrogen, (C₁-C₆)alkyl, (C₂-C₆)alkenyl or (C₂-C₆)alkynyl, where the three last mentioned radicals are substituted by v_{D} radicals from the group consisting of halogen, hydroxy, (C₁-C₄)alkyl, (C₁-C₄)alkoxy and (C₁-C₄)alkylthio, or
R_{D}⁵ and R_{D}⁶ together with the nitrogen atom carrying them form a pyrrolidinyl or piperidinyl radical;
R_{D}⁷ is hydrogen, (C₁-C₄)alkylamino, di-(C₁-C₄)alkylamino, (C₁-C₆)alkyl, (C₃-C₆)cycloalkyl, where the 2 last mentioned radicals are substituted by v_{D} substituents from the group consisting of halogen, (C₁-C₄)alkoxy, halo(C₁-C₆)alkoxy and (C₁-C₄)alkylthio and, in the case of cyclic radicals, also (C₁-C₄)alkyl and (C₁-C₄)haloalkyl;
n_{D} is 0, 1 or 2;
m_{D} is 1 or 2;
v_{D} is 0, 1, 2 or 3;
   from among these, preference is given to compounds of the N-acylsulfonamide type (S4^{a}), for example of the formula below, which are known, for example, from WO-A-97/45016

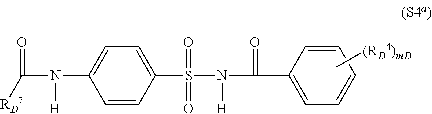

in which
R_{D}⁷ is (C₁-C₆)alkyl, (C₃-C₆)cycloalkyl, where the 2 last mentioned radicals are substituted by v_{D} substituents from the group consisting of halogen, (C₁-C₄)alkoxy, halo(C₁-C₆)alkoxy and (C₁-C₄)alkylthio and, in the case of cyclic radicals, also (C₁-C₄)alkyl and (C₁-C₄)haloalkyl;
R_{D}⁴ is halogen, (C₁-C₄)alkyl, (C₁-C₄)alkoxy, CF₃;
m_{D} is 1 or 2;
v_{D} is 0, 1, 2 or 3;
   and also
   acylsulfamoylbenzamides, for example of the formula (S4^{b}) below, which are known, for example, from WO-A-99/16744,

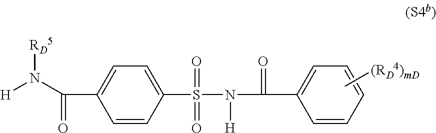

for example those in which
R_{D}⁵=cyclopropyl and (R_{D}⁴)=2-OMe (“cyprosulfamide”, S4-1),
R_{D}⁵=cyclopropyl and (R_{D}⁴)=5-Cl-2-OMe (S4-2),
R_{D}⁵=ethyl and (R_{D}⁴)=2-OMe (S4-3),
R_{D}⁵=isopropyl and (R_{D}⁴)=5-C₁₋₂-OMe (S4-4) and
R_{D}⁵=isopropyl and (R_{D}⁴)=2-OMe (S4-5);
   and also
   compounds of the N-acylsulfamoylphenylurea type of the formula (Se), which are known, for example, from EP-A-365484

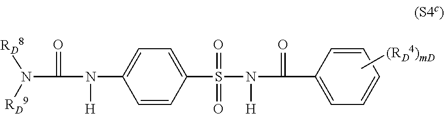

in which
R_{D}⁸ and R_{D}⁹ independently of one another are hydrogen, (C₁-C₈)alkyl, (C₃-C₈)cycloalkyl, (C₃-C₆)alkenyl, (C₃-C₆)alkynyl,
R_{D}⁴ is halogen, (C₁-C₄)alkyl, (C₁-C₄)alkoxy, CF₃
m_{D} is 1 or 2;
   from among these in particular
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea.
S5) Active substances from the class of the hydroxyaromatics and aromatic-aliphatic carboxylic acid derivatives (S5), for example ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active substances from the class of the 1,2-dihydroquinoxalin-2-ones (S6), for example
   1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds of the formula (S7), as described in WO-A-1998/38856

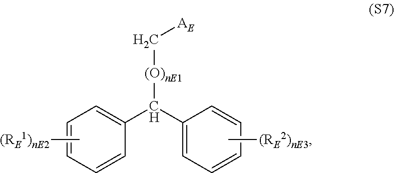

in which the symbols and indices have the following meanings:
R_{E}¹, R_{E}² independently of one another are halogen, (C₁-C₄)alkyl, (C₁-C₄)alkoxy, (C₁-C₄)haloalkyl, (C₁-C₄)alkylamino, di-(C₁-C₄)alkylamino, nitro;
A_{E} is COOR_{E}³ or COSR_{E}⁴
R_{E}³, R_{E}⁴ independently of one another are hydrogen, (C₁-C₄)alkyl, (C₂-C₆)alkenyl, (C₂-C₄)alkynyl, cyanoalkyl, (C₁-C₄)haloalkyl, phenyl, nitrophenyl, benzyl, halobenzyl, pyridinylalkyl and alkylammonium,
n_{E} is 0 or 1
n_{E}², n_{E}³ independently of one another are 0, 1 or 2,
   preferably:
   diphenylmethoxy acetic acid,
   ethyl diphenylmethoxy acetate,
   methyl diphenylmethoxy acetate (CAS-Reg. No. 41858-19-9) (S7-1).
S8) Compounds of the formula (S8) as described in WO-A-98/27049

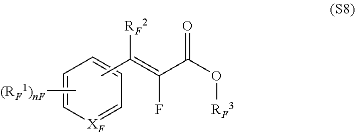

in which
X_{F} is CH or N,
n_{F} in the event that X_{F}=N, is an integer from 0 to 4 and in the event that X_{F}=CH, is an integer from 0 to 5,
R_{F}¹ is halogen, (C₁-C₄)alkyl, (C₁-C₄)haloalkyl, (C₁-C₄)alkoxy, (C₁-C₄)haloalkoxy, nitro, (C₁-C₄)alkylthio, (C₁-C₄)-alkylsulfonyl, (C₁-C₄)alkoxycarbonyl, optionally substituted phenyl, optionally substituted phenoxy,
R_{F}² is hydrogen or (C₁-C₄)alkyl,
R_{F}³ is hydrogen, (C₁-C₈)alkyl, (C₂-C₄)alkenyl, (C₂-C₄)alkynyl, or aryl, each of the abovementioned C-comprising radicals being unsubstituted or substituted by one or more, preferably up to three, identical or different radicals selected from the group consisting of halogen and alkoxy, or their salts,
   preferably compounds in which
X_{F} is CH,
n_{F} is an integer from 0 to 2,
R_{F}¹ is halogen, (C₁-C₄)alkyl, (C₁-C₄)haloalkyl, (C₁-C₄)alkoxy, (C₁-C₄)haloalkoxy,
R_{F}² is hydrogen or (C₁-C₄)alkyl,
R_{F}³ is hydrogen, (C₁-C₈)alkyl, (C₂-C₄)alkenyl, (C₂-C₄)alkynyl, or aryl, where each of the abovementioned C-comprising radicals is unsubstituted or substituted by one or more, preferably up to three, identical or different radicals selected from the group consisting of halogen and alkoxy, or their salts.
S9) Active substances from the class of the 3-(5-tetrazolylcarbonyl)-2-quinolones (S9), for example
   1,2-dihydro-4-hydroxy-1-ethyl-3-(5-tetrazolylcarbonyl)-2-quinolone (CAS-Reg. No. 219479-18-2), 1,2-dihydro-4-hydroxy-1-methyl-3-(5-tetrazolylcarbonyl)-2-quinolone (CAS-Reg. No. 95855-00-8), as described in WO-A-1999/000020.
S10) Compounds of the formulae (S10^{a}) or (S10^{b})
   as described in WO-A-2007/023719 and WO-A-2007/023764

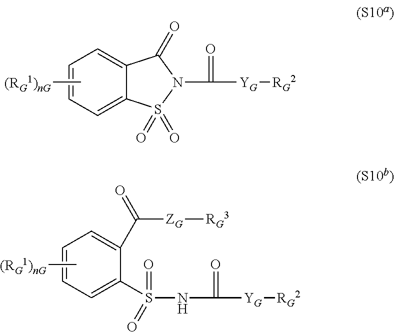

in which
R_{G}¹ is halogen, (C₁-C₄)alkyl, methoxy, nitro, cyano, CF₃, OCF₃
Y_{G}, Z_{G} independently of one another are O or S,
n_{G} is an integer from 0 to 4,
R_{G}² is (C₁-C₁₆)alkyl, (C₂-C₆)alkenyl, (C₃-C₆)cycloalkyl, aryl; benzyl, halobenzyl,
R_{G}³ is hydrogen or (C₁-C₆)alkyl.
S11) Active substances of the oxyimino compound type (S11), which are known as seed-dressing agents, such as, for example,
   “oxabetrinil” ((Z)-1,3-dioxolan-2-ylmethoxyimino(phenyl)acetonitrile) (S11-1), which is known as seed-dressing safener for millet/sorghum against metolachlor-induced damage,
   “fluxofenim” (1-(4-chlorophenyl)-2,2,2-trifluoro-1-ethanone O-(1,3-dioxolan-2-ylmethyl) oxime) (S11-2), which is known as seed-dressing safener for millet/sorghum against metolachlor-induced damage, and
   “cyometrinil” or “CGA-43089” ((Z)-cyanomethoxyimino(phenyl)acetonitrile) (S11-3), which is known as seed-dressing safener for millet/sorghum against metolachlor-induced damage.
S12) Active substances from the class of the isothiochromanones (S12), such as, for example, methyl [(3-oxo-1H-2-benzothiopyran-4(3H)-ylidene)methoxy]acetate (CAS-Reg. No. 205121-04-6) (S12-1) and related compounds from WO-A-1998/13361.
S13) One or more compounds selected from the group (S13) consisting of:
   “naphthalic anhydride” (1,8-naphthalenedicarboxylic anhydride) (S13-1), which is known as seed-dressing safener for maize against thiocarbamate-herbicide-induced damage,
   “fenclorim” (4,6-dichloro-2-phenylpyrimidine) (S13-2), which is known as safener for pretilachlor in seeded rice,
   “flurazole” (benzyl 2-chloro-4-trifluoromethyl-1,3-thiazole-5-carboxylate) (S13-3), which is known as seed-dressing safener for millet/sorghum against alachlor and metolachlor-induced damage,
   “CL 304415” (CAS-Reg. No. 31541-57-8) (4-carboxy-3,4-dihydro-2H-1-benzopyrane-4-acetic acid) (S13-4) from American Cyanamide, which is known as safener for maize against damage induced by imidazolinones,
   “MG 191” (CAS-Reg. No. 96420-72-3) (2-dichloromethyl-2-methyl-1,3-dioxolane) (S13-5) from Nitrokemia, which is known as safener for maize,
   “MG-838” (CAS-Reg. No. 133993-74-5) (2-propenyl 1-oxa-4-azaspiro[4.5]decane-4-carbodithioate) (S13-6) from Nitrokemia,
   “disulfoton” (O,O-diethyl S-2-ethylthioethyl phosphorodithioate) (S13-7),
   “dietholate” (O,O-diethyl O-phenyl phosphorothioate) (S13-8),
   “mephenate” (4-chlorophenyl methylcarbamate) (S13-9).
S14) Active substances which, in addition to a herbicidal action against harmful plants, also have safener action on crop plants such as rice, such as, for example,
   “dimepiperate” or “MY-93” (S-1-methyl-1-phenylethyl piperidine-1-carbothioate), which is known as safener for rice against damage induced by the herbicide molinate,
   “daimuron” or “SK 23” (1-(1-methyl-1-phenylethyl)-3-p-tolylurea), which is known as safener for rice against damage induced by the herbicide imazosulfuron,
   “cumyluron”=“JC-940” (3-(2-chlorophenylmethyl)-1-(1-methyl-1-phenylethyl)urea, see JP-A-60087254), which is known as safener for rice against damage induced by a number of herbicides,
   “methoxyphenone” or “NK 049” (3,3′-dimethyl-4-methoxybenzohenone), which is known as safener for rice against damage induced by a number of herbicides,
   “CSB” (1-bromo-4-(chloromethylsulfonyl)benzene) (CAS Reg. No. 54091-06-4 from Kumiai), which is known as safener against damage induced by a number of herbicides in rice.
S15) Active substances which are employed predominantly as herbicides, but which also have a safener action on crop plants, for example
   (2,4-dichlorophenoxy)acetic acid (2,4-D),
   (4-chlorophenoxy)acetic acid,
   (R,S)-2-(4-chloro-o-tolyloxy)propionic acid (mecoprop),
   4-(2,4-dichlorophenoxy)butyric acid (2,4-DB),
   (4-chloro-o-tolyloxy)acetic acid (MCPA),
   4-(4-chloro-o-tolyloxy)butyric acid,
   4-(4-chlorophenoxy)butyric acid,
   3,6-dichloro-2-methoxybenzoic acid (dicamba),
   1-(ethoxycarbonyl)ethyl 3,6-dichloro-2-methoxybenzoate (lactidichloroethyl).

Preferred herbicide/safener combinations are those which comprise (A) a herbicidally active amount of one or more compounds of the formula (I) or their salts, and (B) an antidote-effective amount of one or more safeners.

For the purposes of the invention, a herbicidally active amount means an amount of one or more herbicides which is suitable for exerting a negative effect on the growth of plants. For the purposes of the invention, an antidote-effective amount means an amount of one or more safeners which is suitable for reducing the phytotoxic effect of plant protection actives (for example of herbicides) on crop plants.

Some of the safeners are already known as herbicides and thus exert not only a herbicidal effect on harmful plants, but also a protective effect on the crop plants.

The weight ratio of herbicide (mixture) to safener will generally depend on the application rate of herbicide and on the efficacy of the safener in question and can vary within wide limits, for example in the range of from 200:1 to 1:200, preferably 100:1 to 1:100, in particular 20:1 to 1:20. The safeners can be formulated together with further herbicides/pesticides analogously to the compounds of the formula (I) or their mixtures and provided, and used, as a ready mix or a tank mix together with the herbicides.

For use, the formulations, which are present in commercially available form, are, if appropriate, diluted in the customary manner, for example by means of water in the case of wettable powders, emulsifiable concentrates, dispersions and water-dispersible granules. Preparations in the form of dusts, soil granules or granules for broadcasting and sprayable solutions are usually not diluted any further with other inert substances prior to use.

The application rate required, for the compounds of the formula (I), varies as a function of the external conditions such as temperature, humidity, the nature of the herbicide used and others. It can vary within wide limits, for example between 0.001 and 10.0 kg/ha or more of active substance, but it is preferably between 0.005 and 5 kg/ha.

The present invention is illustrated in greater detail with reference to the examples which follow which, however, impose no limitation whatsoever on the invention.

### A. Synthesis Examples

### 1. 4-(5,6-Dihydro-1,4,2-dioxazin-3-yl)-N-[(4,6-dimethoxypyrimidin-2-yl)carbamoyl]pyridine-3-sulfonamide (Ia-2)

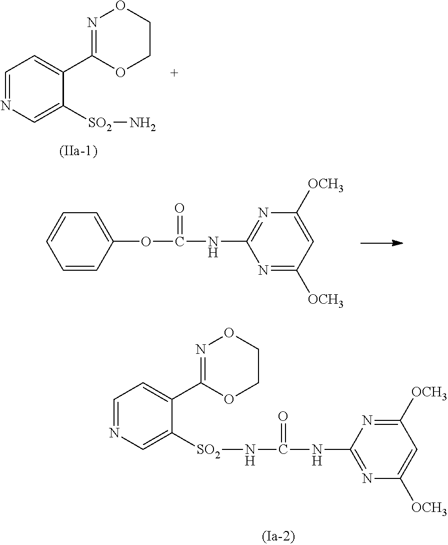

4.9 g (0.02 mol) of 4-(5,6-dihydro-1,4,2-dioxazin-3-yl)pyridine-3-sulfonamide (IIa-1) are dissolved in 40 ml of acetonitrile and treated with 3.1 g (0.02 mol) of DBU (diazabicycloundecene). The clear solution is treated with 5.5 g (0.02 mol) of phenyl (4,6-dimethoxypyrimidin-2-yl)carbamate and the mixture is stirred for 30 min at room temperature. The mixture is poured into 400 ml of aqueous dilute hydrochloric acid, with stirring, and this mixture is stirred thoroughly. The crystals which have precipitated are filtered off with suction and washed with water until the reaction is neutral. The crystals are washed with isopropanol until free from water and then recrystallized from isopropanol. This gives 7.9 g (0.0175 mol) of 4-(5,6-dihydro-1,4,2-dioxazin-3-yl)-N-[(4,6-dimethoxypyrimidin-2-yl)carbamoyl]pyridine-3-sulfonamide of purity (HPLC) 93.9%.

### N-[(4-Chloro-6-methoxypyrimidin-2-yl)carbamoyl]-4-(5,6-dihydro-1,4,2-dioxazin-3-yl)pyridine-3-sulfonamide (Ia-1)

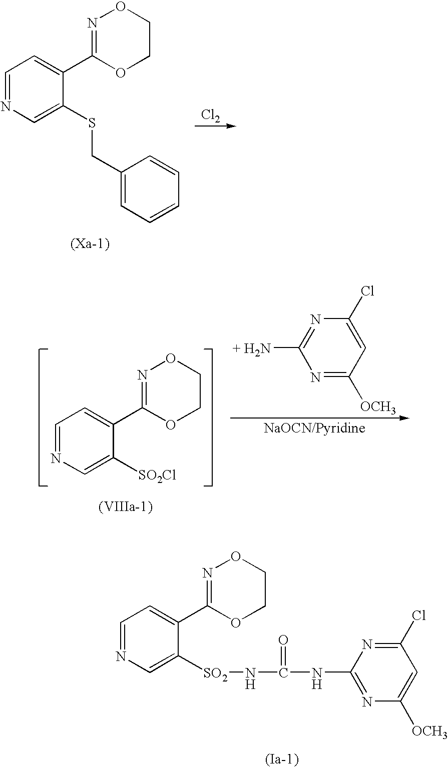

2.0 g (7 mmol) of 3-[3-(benzylsulfanyl)pyridin-4-yl]-5,6-dihydro-1,4,2-dioxazine (Xa-1) are dissolved in 50 ml of dichloromethane and treated with 6.3 g (0.04 mol) of sodium dihydrogen phosphate monohydrate, dissolved in 30 ml of water. Chlorine gas is passed in at 0° C. until a pronounced yellow coloration can be discerned. After the mixture has been stirred for a further 15 min, the organic phase is separated off, washed twice with ice-water, dried and concentrated.

The residue (crude sulfochloride (VIIIa-1)) is dissolved in 5 ml of acetonitrile and added dropwise to a solution of 560 mg (3.5 mmol) of 4-chloro-6-methoxypyrimidine-2-amine, 560 mg (7 mmol) of pyridine and 460 mg (7 mmol) of sodium cyanate in 30 ml of acetonitrile. The reaction mixture is stirred for 16 hours at 20° C., then poured into 500 ml of ice-water and rendered weakly acidic with dilute hydrochloric acid. The mixture is stirred thoroughly and the crystals which have precipitated are filtered off with suction. The crystals are first washed with water and then triturated with isopropanol, again filtered off with suction and washed with isopropanol and diethyl ether. After drying, the product is chromatographed over a silica gel column (methylene chloride/methanol=9/1 volume ratio). This gives 66 mg (0.148 mmol) of N-[(4-chloro-6-methoxypyrimidin-2-yl)carbamoyl]-4-(5,6-dihydro-1,4,2-dioxazin-3-yl)pyridine-3-sulfonamide of purity (HPLC) 96.2%.

### 4-(5,6-Dihydro-1,4,2-dioxazin-3-yl)-N-[(4,6-dimethoxy-1,3,5-triazin-2-yl)carbamoyl]pyridine-3-sulfonamide (Ia-6)

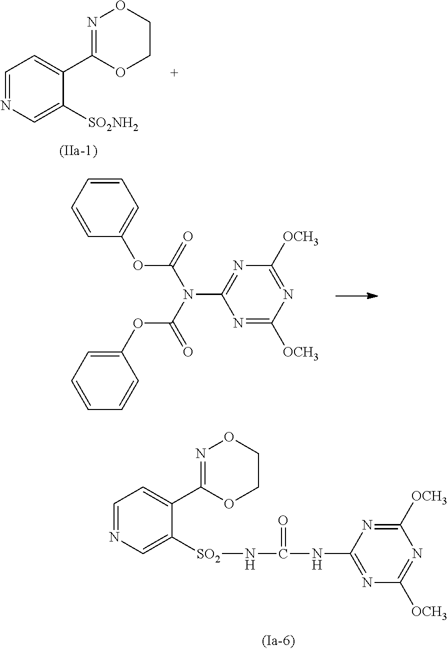

490 mg (2 mmol) of 4-(5,6-dihydro-1,4,2-dioxazin-3-yl)pyridine-3-sulfonamide (IIa-1) are dissolved in 20 ml of acetonitrile at 20° C. and treated with 310 mg (2 mmol) of DBU (diazabicycloundecene). 800 mg (2 mmol) of diphenyl (4,6-dimethoxy-1,3,5-triazin-2-yl)imidodicarbonate (cf. WO 1996/022284) are added and the mixture is stirred for 30 min. Then, the mixture is poured into a mixture of methylene chloride/aqueous hydrochloric acid, with stirring, and the organic phase is separated off. It is washed twice with water and with saturated sodium chloride solution, dried and concentrated. The residue is crystallized with diethyl ether, filtered off with suction and triturated with isopropanol. After the filtration with suction, the residue is washed with diethyl ether and dried. This gives 530 mg (1.14 mmol) of 4-(5,6-dihydro-1,4,2-dioxazin-3-yl)-N-[(4,6-dimethoxy-1,3,5-triazin-2-yl)carbamoyl]pyridine-3-sulfonamide with a purity (HPLC) of 91.3%.

The compounds of the formula (I) described in the tables hereinbelow are obtained in accordance with, or analogously, to the synthesis examples described hereinabove:

TABLE 1a
Compounds of the formula (Ia)
(Ia)

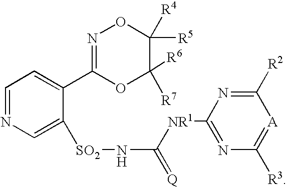

| Ex. No. | R¹ | R² | R³ | R⁴ | R⁶ | R⁵ | R⁷ | Q | A |
|---|---|---|---|---|---|---|---|---|---|
| Ia-1 | H | Cl | OCH₃ | H | H | H | H | O | CH |
| Ia-2 | H | OCH₃ | OCH₃ | H | H | H | H | O | CH |
| Ia-3 | H | CH₃ | CH₃ | H | H | H | H | O | CH |
| Ia-4 | H | SCH₃ | OCH₃ | H | H | H | H | O | CH |
| Ia-5 | H | H | OCH₃ | H | H | H | H | O | CH |
| Ia-6 | H | OCH₃ | OCH₃ | H | H | H | H | O | N |
| Ia-7 | H | CH₃ | OCH₃ | H | H | H | H | O | N |
| Ia-8 | H | N(CH₃)₂ | OCH₂CF₃ | H | H | H | H | O | N |
| Ia-9 | H | CH₃ | OCH₃ | H | H | H | H | O | CH |
| Ia-10 | H | OCH₃ | OCH₃ | CH(CH₃)₂ | H | H | H | O | CH |
| Ia-11 | H | OCH₃ | OCH₃ | CH₃ | H | H | H | O | CH |
| Ia-12 | H | CH₃ | OCH₃ | CH₃ | H | CH₃ | H | O | N |
| Ia-13 | H | OCH₃ | OCH₃ | CH₃ | H | CH₃ | H | O | CH |
| Ia-14 | H | Cl | OCH₃ | CH₃ | H | CH₃ | H | O | CH |
| Ia-15 | H | CH₃ | OCH₃ | CH₃ | H | H | H | O | CH |
| Ia-16 | H | Cl | OCH₃ | CH₃ | H | H | H | O | CH |
| Ia-17 | H | CH₃ | OCH₃ | CH₂CH₂CH₃ | H | H | H | O | CH |
| Ia-18 | H | CH₃ | OCH₃ | CH₂CH₃ | H | H | H | O | N |
| Ia-19 | H | CH₃ | OCH₃ | CH₂CH₃ | H | H | H | O | CH |
| la-20 | H | Cl | OCH₃ | CH₂CH₃ | H | H | H | O | CH |
| Ia-21 | H | OCH₃ | OCH₃ | CH₂CH₂CH₃ | H | H | H | O | CH |
| Ia-22 | H | CH₃ | OCH₃ | CH(CH₃)₂ | H | H | H | O | CH |
| Ia-23 | H | CH₃ | OCH₃ | CH₂CH₂CH₃ | H | H | H | O | N |
| Ia-24 | H | OCH₃ | OCH₃ | CH₂CH₃ | H | H | H | O | CH |
| Ia-25 | H | OCH₃ | OCH₃ | H | H | H | H | S | CH |

| | | | | | | | | |
|---|---|---|---|---|---|---|---|---|
| Ia-26 | H | OCH₃ | OCH₃ | CH₂CH₂CH₂ | H | H | O | CH |
| Ia-27 | H | OCH₃ | OCH₃ | CH₂CH₂CH₂CH₂ | H | H | O | CH |
| Ia-28 | H | OCH₃ | OCH₃ | CH₂—O—CH₂ | H | H | O | CH |
| Ia-29 | H | OCH₃ | OCH₃ | CH₂—S—CH₂ | H | H | O | CH |
| Ia-30 | H | OCH₃ | OCH₃ | CH₂—O—CH₂CH₂ | H | H | O | CH |
| Ia-31 | H | OCH₃ | OCH₃ | CH₂—S—CH₂CH₂ | H | H | O | CH |

¹H NMR data (400 MHz, solvent: CD₃CN, internal standard: tetramethylsilane δ=0.00 ppm; s=singulet, br. s=broad singulet, d=doublet, dd=double doublet, m=multiplet, q=quartet, t=triplet)

Ia-1: δ (CDCl₃)=4.01 (s, 3H); 4.27 (m, 2H); 4.58 (m, 2H); 6.50 (s, 1H); 7.54 (d, 1H); 8.91 (d, 1H); 9.52 (br. s, 1H); 12.02 (br. s, 1H) ppm

Ia-2: δ ([D₇]-DMF)=3.99 (s, 6H); 4.30 (m, 2H); 4.60 (m, 2H); 6.00 (s, 1H); 7.80 (dd, 1H); 9.05 (d, 1H); 9.39 (br. s, 1H); 10.80 (s, 1H); 12.91 (s, 1H) ppm

Ia-3: δ (CD₃CN)=2.42 (s, 6H); 4.19 (m, 2H); 4.45 (m, 2H); 6.88 (s, 1H); 7.57 (dd, 1H); 8.09 (br. s, 1H); 8.88 (d, 1H); 9.33 (br. s, 1H); 13.2 (br. s, 1H) ppm

Ia-6: δ (CD₃CN)=4.02 (s, 6H); 4.20 (m, 2H); 4.51 (m, 2H); 7.60 (d, 1H); 8.38 (br. s, 1H); 8.90 (d, 1H); 9.32 (br. s, 1H); 12.3 (br. s, 1H) ppm

Ia-7: δ (CD₃CN)=2.50 (s, 3H); 4.01 (s, 3H); 4.21 (m, 2H); 4.49 (m, 2H); 7.60 (m, 1H); 8.40 (br. s, 1H); 8.90 (d, 1H); 9.33 (br. s, 1H); 12.58 (br. s, 1H) ppm

Ia-8: δ (CD₃CN)=3.17 (s, 6H); 4.18 (m, 2H); 4.47 (m, 2H); 4.86 (q, 2H); 7.57 (d, 1H); 8.87 (d, 1H); 9.31 (br. s, 1H) ppm

Ia-9: δ (CD₃CN)=2.41 (s, 3H); 3.94 (s, 3H); 4.19 (m, 2H); 4.46 (m, 2H); 6.39 (br. s, 1H); 7.57 (d, 1H); 8.25 (br. s, 1H); 8.88 (d, 1H); 9.33 (br. s, 1H) ppm

TABLE Ib
Compounds of the formula (Ib)
(Ib)

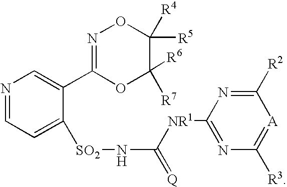

| Ex. No. | R¹ | R² | R³ | R⁴ | R⁶ | R⁵ | R⁷ | Q | A |
|---|---|---|---|---|---|---|---|---|---|
| Ib-1 | H | Cl | OCH₃ | H | H | H | H | O | CH |
| Ib-2 | H | OCH₃ | OCH₃ | H | H | H | H | O | CH |
| Ib-3 | H | CH₃ | CH₃ | H | H | H | H | O | CH |
| Ib-4 | H | SCH₃ | OCH₃ | H | H | H | H | O | CH |
| Ib-5 | H | H | OCH₃ | H | H | H | H | O | CH |
| Ib-6 | H | OCH₃ | OCH₃ | H | H | H | H | O | N |
| Ib-7 | H | CH₃ | OCH₃ | H | H | H | H | O | N |
| Ib-8 | H | N(CH₃)₂ | OCH₂CF₃ | H | H | H | H | O | N |
| Ib-9 | H | CH₃ | OCH₃ | H | H | H | H | O | CH |
| Ib-10 | H | OCH₃ | OCH₃ | CH(CH₃)₂ | H | H | H | O | CH |
| Ib-11 | H | OCH₃ | OCH₃ | CH₃ | H | H | H | O | CH |
| Ib-12 | H | CH₃ | OCH₃ | CH₃ | H | CH₃ | H | O | N |
| Ib-13 | H | OCH₃ | OCH₃ | CH₃ | H | CH₃ | H | O | CH |
| Ib-14 | H | Cl | OCH₃ | CH₃ | H | CH₃ | H | O | CH |
| Ib-15 | H | CH₃ | OCH₃ | CH₃ | H | H | H | O | CH |
| Ib-16 | H | Cl | OCH₃ | CH₃ | H | H | H | O | CH |
| Ib-17 | H | CH₃ | OCH₃ | CH₂CH₂CH₃ | H | H | H | O | CH |
| Ib-18 | H | CH₃ | OCH₃ | CH₂CH₃ | H | H | H | O | N |
| Ib-19 | H | CH₃ | OCH₃ | CH₂CH₃ | H | H | H | O | CH |
| Ib-20 | H | Cl | OCH₃ | CH₂CH₃ | H | H | H | O | CH |
| Ib-21 | H | OCH₃ | OCH₃ | CH₂CH₂CH₃ | H | H | H | O | CH |
| Ib-22 | H | CH₃ | OCH₃ | CH(CH₃)₂ | H | H | H | O | CH |
| Ib-23 | H | CH₃ | OCH₃ | CH₂CH₂CH₃ | H | H | H | O | N |
| Ib-24 | H | OCH₃ | OCH₃ | CH₂CH₃ | H | H | H | O | CH |
| Ib-25 | H | OCH₃ | OCH₃ | H | H | H | H | S | CH |

| | | | | | | | | |
|---|---|---|---|---|---|---|---|---|
| Ib-26 | H | OCH₃ | OCH₃ | CH₂CH₂CH₂ | H | H | O | CH |
| Ib-27 | H | OCH₃ | OCH₃ | CH₂CH₂CH₂CH₂ | H | H | O | CH |
| Ib-28 | H | OCH₃ | OCH₃ | CH₂—O—CH₂ | H | H | O | CH |
| Ib-29 | H | OCH₃ | OCH₃ | CH₂—S—CH₂ | H | H | O | CH |
| Ib-30 | H | OCH₃ | OCH₃ | CH₂—O—CH₂CH₂ | H | H | O | CH |
| Ib-31 | H | OCH₃ | OCH₃ | CH₂—S—CH₂CH₂ | H | H | O | CH |

TABLE 1c
Compounds of the formula (Ic)
(Ic)

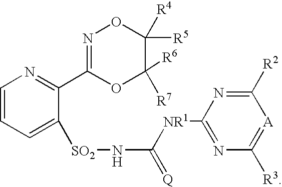

| Ex. No. | R¹ | R² | R³ | R⁴ | R⁶ | R⁵ | R⁷ | Q | A |
|---|---|---|---|---|---|---|---|---|---|
| Ic-1 | H | OCH₃ | OCH₃ | H | H | H | H | O | CH |
| Ic-2 | H | Cl | OCH₃ | H | H | H | H | O | CH |
| Ic-3 | H | CH₃ | CH₃ | H | H | H | H | O | CH |
| Ic-4 | H | SCH₃ | OCH₃ | H | H | H | H | O | CH |
| Ic-5 | H | H | OCH₃ | H | H | H | H | O | CH |
| Ic-6 | H | OCH₃ | OCH₃ | H | H | H | H | O | N |
| Ic-7 | H | CH₃ | OCH₃ | H | H | H | H | O | N |
| Ic-8 | H | N(CH₃)₂ | OCH₂CF₃ | H | H | H | H | O | N |
| Ic-9 | H | CH₃ | OCH₃ | H | H | H | H | O | CH |
| Ic-10 | H | OCH₃ | OCH₃ | CH(CH₃)₂ | H | H | H | O | CH |
| Ic-11 | H | OCH₃ | OCH₃ | CH₃ | H | H | H | O | CH |
| Ic-12 | H | CH₃ | OCH₃ | CH₃ | H | CH₃ | H | O | N |
| Ic-13 | H | OCH₃ | OCH₃ | CH₃ | H | CH₃ | H | O | CH |
| Ic-14 | H | Cl | OCH₃ | CH₃ | H | CH₃ | H | O | CH |
| Ic-15 | H | CH₃ | OCH₃ | CH₃ | H | H | H | O | CH |
| Ic-16 | H | Cl | OCH₃ | CH₃ | H | H | H | O | CH |
| Ic-17 | H | CH₃ | OCH₃ | CH₂CH₂CH₃ | H | H | H | O | CH |
| lc-18 | H | CH₃ | OCH₃ | CH₂CH₃ | H | H | H | O | N |
| Ic-19 | H | CH₃ | OCH₃ | CH₂CH₃ | H | H | H | O | CH |
| Ic-20 | H | Cl | OCH₃ | CH₂CH₃ | H | H | H | O | CH |
| Ic-21 | H | OCH₃ | OCH₃ | CH₂CH₂CH₃ | H | H | H | O | CH |
| Ic-22 | H | CH₃ | OCH₃ | CH(CH₃)₂ | H | H | H | O | CH |
| Ic-23 | H | CH₃ | OCH₃ | CH₂CH₂CH₃ | H | H | H | O | N |
| Ic-24 | H | OCH₃ | OCH₃ | CH₂CH₃ | H | H | H | O | CH |
| Ic-25 | H | OCH₃ | OCH₃ | H | H | H | H | S | CH |

| | | | | | | | | |
|---|---|---|---|---|---|---|---|---|
| Ic-26 | H | OCH₃ | OCH₃ | CH₂CH₂CH₂ | H | H | O | CH |
| Ic-27 | H | OCH₃ | OCH₃ | CH₂CH₂CH₂CH₂ | H | H | O | CH |
| lc-28 | H | OCH₃ | OCH₃ | CH₂—O—CH₂ | H | H | O | CH |
| Ic-29 | H | OCH₃ | OCH₃ | CH₂—S—CH₂ | H | H | O | CH |
| Ic-30 | H | OCH₃ | OCH₃ | CH₂—O—CH₂CH₂ | H | H | O | CH |
| Ic-31 | H | OCH₃ | OCH₃ | CH₂—S—CH₂CH₂ | H | H | O | CH |

¹H NMR data (400 MHz, solvent: CD₃CN, internal standard: tetramethylsilane δ=0.00 ppm; s=singulet, br. s=broad singulet, d=doublet, dd=double doublet, m=multiplet, q=quartet, t=triplet)

Ic-1: δ=3.95 (s, 6H); 4.19 (m, 2H); 4.48 (m, 2H); 5.86 (s, 1H); 7.68 (dd, 1H); 8.29 (br. s, 1H); 8.59 (dd, 1H); 8.85 (dd, 1H); 12.80 (s, 1H) ppm

Ic-2: δ=4.02 (s, 3H); 4.21 (m, 2H); 4.50 (m, 2H); 6.62 (s, 1H); 7.69 (dd, 1H); 8.51 (br. s, 1H); 8.58 (dd, 1H); 8.86 (dd, 1H); 12.25 (s, 1H) ppm

### 2. 4-(5,6-Dihydro-1,4,2-dioxazin-3-yl)pyridine-3-sulfonamide (IIa-1)

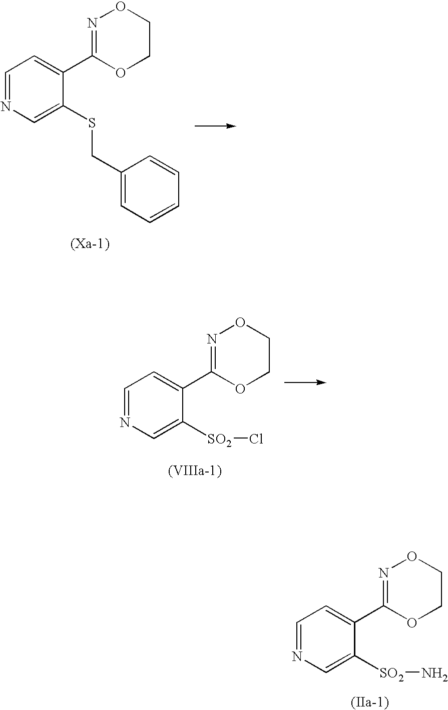

10.6 g (37.1 mmol) of 3-[3-(benzylsulfanyl)pyridin-4-yl]-5,6-dihydro-1,4,2-dioxazine (Xa-1) are dissolved in 85 ml of dichloromethane and treated with a solution of 33.2 g (0.24 mol) of sodium dihydrogen phosphate monohydrate in 70 ml of water. Chlorine gas is passed in swiftly at <10° C., until a pale yellow coloration can be discerned. After stirring of the mixture has been continued for 5 min, the organic phase is separated off and washed twice with in each case 50 ml of ice-water. The cold solution of 4-(5,6-dihydro-1,4,2-dioxazin-3-yl)pyridine-3-sulfonyl chloride (VIIIa-1) is added dropwise at <10° C. to 10 ml of briskly stirred 25% strength ammonia solution. The mixture is allowed to come to room temperature, vigorous stirring is continued for 1 h. Then, the mixture is cooled to 0° C. and the resulting precipitation is filtered off with suction and washed with ice-water and 10 ml of cold methyl tert-butyl ether. It is stirred with diethyl ether and filtered off with suction. This gives 7.1 g (28.7 mmol) of 4-(5,6-dihydro-1,4,2-dioxazin-3-yl)pyridine-3-sulfonamide (IIa-1) with a purity (HPLC) of 98.4%, m.p.: 163-164° C.

The following can be prepared analogously:

TABLE 2a
Compounds of the formula (VIIIa)
(VIIIa)

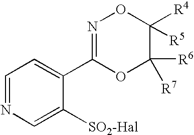

| Ex. No. | R⁴ | R⁶ | R⁵ | R⁷ | Hal |
|---|---|---|---|---|---|
| VIIIa-1 | H | H | H | H | Cl |
| VIIIa-2 | CH(CH₃)₂ | H | H | H | Cl |
| VIIIa-3 | CH₃ | H | H | H | Cl |
| VIIIa-4 | CH₃ | H | CH₃ | H | Cl |
| VIIIa-5 | CH₂CH₂CH₃ | H | H | H | Cl |
| VIIIa-6 | CH₂CH₃ | H | H | H | Cl |
| VIIIa-7 | CH₃ | CH₃ | H | H | Cl |

| | | | | |
|---|---|---|---|---|
| VIIIa-8 | CH₂CH₂CH₂ | H | H | Cl |
| VIIIa-9 | CH₂CH₂CH₂CH₂ | H | H | Cl |
| VIIIa-10 | CH₂—O—CH₂ | H | H | Cl |
| VIIIa-11 | CH₂—S—CH₂ | H | H | Cl |
| VIIIa-12 | CH₂—O—CH₂CH₂ | H | H | Cl |
| VIIIa-13 | CH₂—S—CH₂CH₂ | H | H | Cl |

TABLE 2b
Compounds of the formula (VIIIb)
(VIIIb)

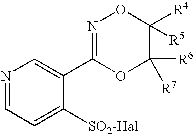

| Ex. No. | R⁴ | R⁶ | R⁵ | R⁷ | Hal |
|---|---|---|---|---|---|
| VIIIb-1 | H | H | H | H | Cl |
| VIIIb-2 | CH(CH₃)₂ | H | H | H | Cl |
| VIIIb-3 | CH₃ | H | H | H | Cl |
| VIIIb-4 | CH₃ | H | CH₃ | H | Cl |
| VIIIb-5 | CH₂CH₂CH₃ | H | H | H | Cl |
| VIIIb-6 | CH₂CH₃ | H | H | H | Cl |
| VIIIb-7 | CH₃ | CH₃ | H | H | Cl |

| | | | | |
|---|---|---|---|---|
| VIIIb-8 | CH₂CH₂CH₂ | H | H | Cl |
| VIIIb-9 | CH₂CH₂CH₂CH₂ | H | H | Cl |
| VIIIb-10 | CH₂—O—CH₂ | H | H | Cl |
| VIIIb-11 | CH₂—S—CH₂ | H | H | Cl |
| VIIIb-12 | CH₂—O—CH₂CH₂ | H | H | Cl |
| VIIIb-13 | CH₂—S—CH₂CH₂ | H | H | Cl |

TABLE 2c
Compounds of the formula (VIIIc)
(VIIIc)

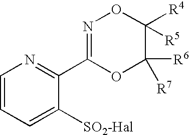

| Ex. No. | R⁴ | R⁶ | R⁵ | R⁷ | Hal |
|---|---|---|---|---|---|
| VIIIc-1 | H | H | H | H | Cl |
| VIIIc-2 | CH(CH₃)₂ | H | H | H | Cl |
| VIIIc-3 | CH₃ | H | H | H | Cl |
| VIIIc-4 | CH₃ | H | CH₃ | H | Cl |
| VIIIc-5 | CH₂CH₂CH₃ | H | H | H | Cl |
| VlIIc-6 | CH₂CH₃ | H | H | H | Cl |
| VIIIc-7 | CH₃ | CH₃ | H | H | Cl |

| | | | | |
|---|---|---|---|---|
| VIIIc-8 | CH₂CH₂CH₂ | H | H | Cl |
| VIIIc-9 | CH₂CH₂CH₂CH₂ | H | H | Cl |
| VIIIc-10 | CH₂—O—CH₂ | H | H | Cl |
| VIIIc-11 | CH₂—S—CH₂ | H | H | Cl |
| VIIIc-12 | CH₂—O—CH₂CH₂ | H | H | Cl |
| VIIIc-13 | CH₂—S—CH₂CH₂ | H | H | Cl |

TABLE 3a
Compounds of the formula (IIa)
(IIa)

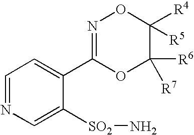

| Ex. No. | R⁴ | R⁶ | R⁵ | R⁷ |
|---|---|---|---|---|
| IIa-1 | H | H | H | H |
| IIa-2 | CH(CH₃)₂ | H | H | H |
| IIa-3 | CH₃ | H | H | H |
| IIa-4 | CH₃ | H | CH₃ | H |
| IIa-5 | CH₂CH₂CH₃ | H | H | H |
| IIa-6 | CH₂CH₃ | H | H | H |
| IIa-7 | CH₃ | CH₃ | H | H |

| | | | |
|---|---|---|---|
| IIa-8 | CH₂CH₂CH₂ | H | H |
| IIa-9 | CH₂CH₂CH₂CH₂ | H | H |
| IIa-10 | CH₂—O—CH₂ | H | H |
| IIa-11 | CH₂—S—CH₂ | H | H |
| IIa-12 | CH₂—O—CH₂CH₂ | H | H |
| IIa-13 | CH₂—S—CH₂CH₂ | H | H |

Melting Point:

IIa-1: 163-164° C.

TABLE 3b
Compounds of the formula (IIb)
(IIb)

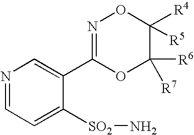

| Ex. No. | R⁴ | R⁶ | R⁵ | R⁷ |
|---|---|---|---|---|
| IIb-1 | H | H | H | H |
| IIb-2 | CH(CH₃)₂ | H | H | H |
| IIb-3 | CH₃ | H | H | H |
| IIb-4 | CH₃ | H | CH₃ | H |
| IIb-5 | CH₂CH₂CH₃ | H | H | H |
| IIb-6 | CH₂CH₃ | H | H | H |
| IIb-7 | CH₃ | CH₃ | H | H |

| | | | |
|---|---|---|---|
| IIb-8 | CH₂CH₂CH₂ | H | H |
| IIb-9 | CH₂CH₂CH₂CH₂ | H | H |
| IIb-10 | CH₂—O—CH₂ | H | H |
| Ilb-11 | CH₂—S—CH₂ | H | H |
| IIb-12 | CH₂—O—CH₂CH₂ | H | H |
| IIb-13 | CH₂—S—CH₂CH₂ | H | H |

TABLE 3c
Compounds of the formula (IIc)
(IIc)

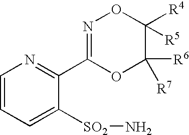

| Ex. No. | R⁴ | R⁶ | R⁵ | R⁷ |
|---|---|---|---|---|
| IIc-1 | H | H | H | H |
| IIc-2 | CH(CH₃)₂ | H | H | H |
| IIc-3 | CH₃ | H | H | H |
| IIc-4 | CH₃ | H | CH₃ | H |
| IIc-5 | CH₂CH₂CH₃ | H | H | H |
| IIc-6 | CH₂CH₃ | H | H | H |
| IIc-7 | CH₃ | CH₃ | H | H |

| | | | |
|---|---|---|---|
| IIc-8 | CH₂CH₂CH₂ | H | H |
| IIc-9 | CH₂CH₂CH₂CH₂ | H | H |
| IIc-10 | CH₂—O—CH₂ | H | H |
| IIc-11 | CH₂—S—CH₂ | H | H |
| IIc-12 | CH₂—O—CH₂CH₂ | H | H |
| IIc-13 | CH₂—S—CH₂CH₂ | H | H |

### 3. 3-[3-(Benzylsulfanyl)pyridin-4-yl]-5,6-dihydro-1,4,2-dioxazine (Xa-1)

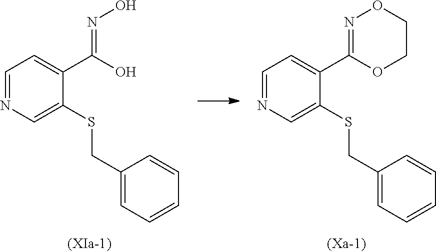

13.0 g (50 mmol) of 3-(benzylsulfanyl)-N-hydroxypyridine-4-carboximic acid (XIa-1) are taken up in 60 ml of water and 60 ml of ethanol and the mixture is treated with 39 g (0.28 mol) of potassium carbonate powder. 13.5 g (61.5 mmol) of ethane-1,2-diyl dimethanesulfonate are added with stirring. Then, the mixture is stirred for 16 h at 40° C., during which process two phases are formed. Upon cooling, crystals precipitate. The reaction mixture is diluted with 100 ml of water, cooled to 5° C. and filtered. The crystals are filtered off with suction and then washed first with water and subsequently with isopropanol and diethyl ether. This gives 11.0 g (38.4 mmol) of 3-[3-(Benzylsulfanyl)pyridin-4-yl]-5,6-dihydro-1,4,2-dioxazine with a purity (HPLC) of 100%, m.p.: 110-112° C.

The following can be prepared analogously:

TABLE 4a
Compounds of the formula (Xa)
(Xa)

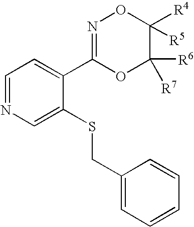

| Ex. No. | R⁴ | R⁶ | R⁵ | R⁷ |
|---|---|---|---|---|
| Xa-1 | H | H | H | H |
| Xa-2 | CH₃ | H | H | H |
| Xa-3 | CH(CH₃)₂ | H | H | H |
| Xa-4 | CH₃ | H | CH₃ | H |
| Xa-5 | CH₂CH₂CH₃ | H | H | H |
| Xa-6 | CH₂CH₃ | H | H | H |
| Xa-7 | CH₃ | CH₃ | H | H |

| | | | |
|---|---|---|---|
| Xa-8 | CH₂CH₂CH₂ | H | H |
| Xa-9 | CH₂CH₂CH₂CH₂ | H | H |
| Xa-10 | CH₂—O—CH₂ | H | H |
| Xa-11 | CH₂—S—CH₂ | H | H |
| Xa-12 | CH₂—O—CH₂CH₂ | H | H |
| Xa-13 | CH₂—S—CH₂CH₂ | H | H |

Melting Point:

Xa-1: 110-112° C.

TABLE 4b
Compounds of the formula (Xb)
(Xb)

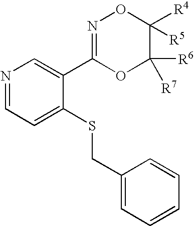

| Ex. No. | R⁴ | R⁶ | R⁵ | R⁷ |
|---|---|---|---|---|
| Xb-1 | H | H | H | H |
| Xb-2 | CH₃ | H | H | H |
| Xb-3 | CH(CH₃)₂ | H | H | H |
| Xb-4 | CH₃ | H | CH₃ | H |
| Xb-5 | CH₂CH₂CH₃ | H | H | H |
| Xb-6 | CH₂CH₃ | H | H | H |
| Xb-7 | CH₃ | CH₃ | H | H |

| | | | |
|---|---|---|---|
| Xb-8 | CH₂CH₂CH₂ | H | H |
| Xb-9 | CH₂CH₂CH₂CH₂ | H | H |
| Xb-10 | CH₂—O—CH₂ | H | H |
| Xb-11 | CH₂—S—CH₂ | H | H |
| Xb-12 | CH₂—O—CH₂CH₂ | H | H |
| Xb-13 | CH₂—S—CH₂CH₂ | H | H |

Melting Point:

Xb-1: 150-151° C.

TABLE 4c
Compounds of the formula (Xc)
(Xc)

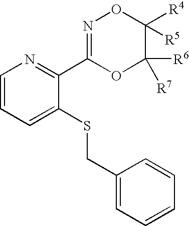

| Ex. No. | 5R⁴ | R⁶ | R⁵ | R⁷ |
|---|---|---|---|---|
| Xc-1 | H | H | H | H |
| Xc-2 | CH₃ | H | H | H |
| Xc-3 | CH(CH₃)₂ | H | H | H |
| Xc-4 | CH₃ | H | CH₃ | H |
| Xc-5 | CH₂CH₂CH₃ | H | H | H |
| Xc-6 | CH₂CH₃ | H | H | H |
| Xc-7 | CH₃ | CH₃ | H | H |

| | | | |
|---|---|---|---|
| Xc-8 | CH₂CH₂CH₂ | H | H |
| Xc-9 | CH₂CH₂CH₂CH₂ | H | H |
| Xc-10 | CH₂—O—CH₂ | H | H |
| Xc-11 | CH₂—S—CH₂ | H | H |
| Xc-12 | CH₂—O—CH₂CH₂ | H | H |
| Xc-13 | CH₂—S—CH₂CH₂ | H | H |

¹H NMR data (400 MHz, solvent: CD₃CN, internal standard: tetramethylsilane δ=0.00 ppm; s=singulet, d=doublet, m=multiplet)

Xc-1: δ=4.17 (m, 2H); 4.18 (s, 2H); 4.47 (m, 2H); 7.18-7.39 (m, 6H); 7.80 (d, 1H); 8.38 (m, 1H) ppm

### 4. 3-(Benzylsulfanyl)-N-hydroxypyridine-4-carboximic Acid (XIa-1)

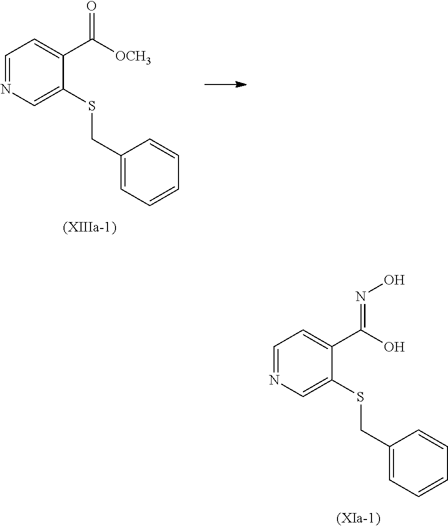

23.4 g (90 mmol) of methyl 3-(benzylsulfanyl)isonicotinate (XIIIa-1) together with 12.5 g (180 mmol) of hydroxylamine hydrochloride are taken up in 200 ml of methanol. With stirring, 76.2 g of a 30% strength sodium methoxide solution are swiftly added dropwise at <5° C. The mixture is allowed to come to room temperature and stirring is continued for 16 h. The mixture is poured into 600 ml of ice-water and rendered weakly acidic using concentrated hydrochloric acid, during which process it is stirred vigorously. The crystals formed are filtered off with suction and washed first with water and then with cold isopropanol. After the crystals have been stirred with diisopropyl ether, they are filtered off with suction and dried. This gives 17.8 g (64.3 mmol) of 3-(benzylsulfanyl)-N-hydroxypyridine-4-carboximic acid with a purity (HPLC) of 94.1%, m.p.: 148° C.

The following can be prepared analogously:

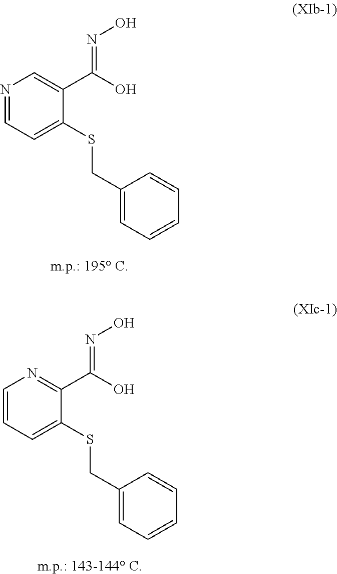

### 5. Methyl 3-(benzylsulfanyl)isonicotinate (XIIIa-1)

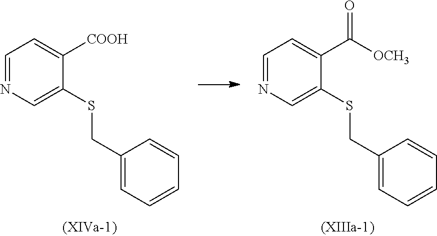

27.7 g (0.11 mol) of 3-(benzylsulfanyl)isonicotinic acid (XIVa-1) are refluxed in 150 ml of thionyl chloride until the evolution of gas has ceased and a clear solution has formed. 100 ml of ethanol are added dropwise, during which process HCl is evolved vigorously and the temperature of the mixture rises drastically. After the mixture has been refluxed for 15 min, it is concentrated, and the residue is taken up in methylene chloride and washed with saturated sodium hydrogen carbonate solution. After drying and concentrating, the residue is stirred in diisopropyl ether and filtered off with suction. This gives 22.6 g (86.5 mmol) of methyl 3-(benzylsulfanyl)isonicotinate with a purity (HPLC) of 99.2%, m.p.: 80° C.

The following can be prepared analogously:

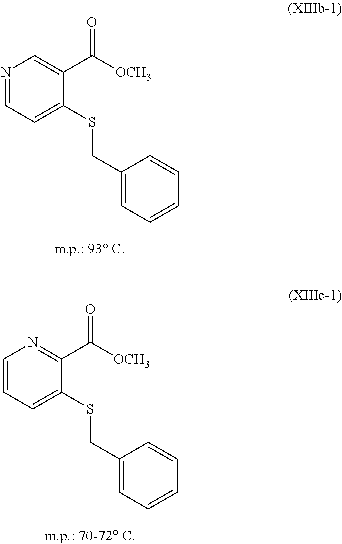

### 6. (Benzylsulfanyl)pyridinecarboxylic Acids (XIV-1)

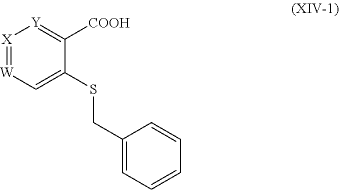

These are synthesized as described in protocols or modified protocols according to U.S. Pat. No. 4,767,766.

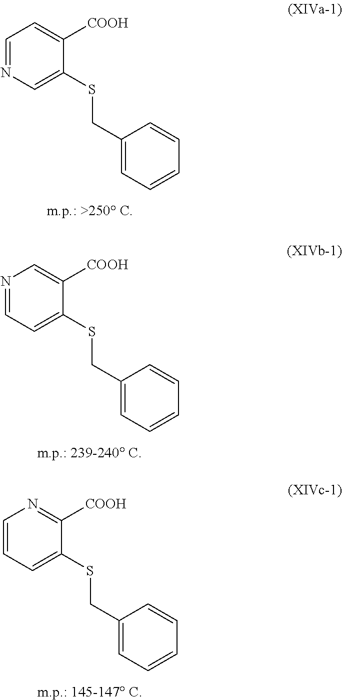

### B. Formulation Examples

a) A dust is obtained by mixing 10 parts by weight of a compound of the formula (I) and 90 parts by weight of talc as inert substance and comminuting the mixture in a hammer mill.
b) A wettable powder which is readily dispersible in water is obtained by mixing 25 parts by weight of a compound of the formula (I), 64 parts by weight of kaolin-containing quartz as inert substance, 10 parts by weight of potassium lignosulfonate and 1 part by weight of sodium oleoylmethyltaurinate as wetting agent and dispersant and grinding the mixture in a pinned-disk mill.
c) A dispersion concentrate which is readily dispersible in water is obtained by mixing 20 parts by weight of a compound of the formula (I) with 6 parts by weight of alkylphenol polyglycol ether (®Triton X 207), 3 parts by weight of isotridecanol polyglycol ether (8 EO) and 71 parts by weight of paraffinic mineral oil (boiling range for example approximately 255 to above 277° C.) and grinding the mixture in a ball mill to a fineness of below 5 micrometers.
d) An emulsifiable concentrate is obtained from 15 parts by weight of a compound of the formula (I), 75 parts by weight of cyclohexanone as solvent and 10 parts by weight of oxethylated nonylphenol as emulsifier.
e) Water-dispersible granules are obtained by mixing

| | |
|---|---|
| 75 parts by weight | of a compound of the formula (I), |
| 10 parts by weight | of calcium lignosulfonate, |
| 5 parts by weight | of sodium lauryl sulfate, |
| 3 parts by weight | of polyvinyl alcohol and |
| 7 parts by weight | of kaolin, |

grinding the mixture in a pinned-disk mill and granulating the powder in a fluidized bed by spraying on water as granulation liquid.
f) Water-dispersible granules are also obtained by homogenizing and precomminuting, in a colloid mill,

| | |
|---|---|
| 25 parts by weight | of a compound of the formula (I), |
| 5 parts by weight | of sodium 2,2′-dinaphthylmethane-6,6′-disulfonate, |
| 2 parts by weight | of sodium oleoylmethyltaurinate, |
| 1 part by weight | of polyvinyl alcohol, |
| 17 parts by weight | of calcium carbonate and |
| 50 parts by weight | of water, |

then grinding the mixture in a bead mill and atomizing and drying the resulting suspension in a spray tower by means of a single-substance nozzle.

### C. Biological Examples

### 1. Pre-Emergence Herbicidal Activity/Crop Plant Tolerance

Seeds of monocotyledonous or dicotyledonous weed plants or crop plants are placed in sandy loam in wood fiber pots and covered with soil. The compounds according to the invention, which are formulated as wettable powders (WP) are then applied to the surface of the soil cover in the form of an aqueous suspension or emulsion with a water application rate of 600 I/ha (converted), with addition of 0.2% wetter.

After the treatment, the pots are placed in the greenhouse and maintained under good growth conditions for the test plants. After approximately 3 weeks, the activity of the preparation is scored visually in comparison with untreated controls (herbicidal activity in percent (%): 100% activity=plants have died, 0% activity=like control plants).

The following results were achieved when using the compounds according to the invention pre-emergence:

| Com- | Dosage | | | | | | | | | | | | | |
|---|---|---|---|---|---|---|---|---|---|---|---|---|---|---|
| pound | rate | Unit | ALOMY | AMARE | CHEAL | DIGSA | ECHCG | LOLMU | MATIN | PHBPU | SETVI | SINAL | STEME | VIOTR |
| Ic-1 | 80 | g/ha | 90 | 90 | | 80 | 90 | 85 | | | 85 | 90 | 90 | 80 |
| Ia-2 | 80 | g/ha | 90 | 90 | 100 | 80 | 90 | 90 | 90 | 80 | 100 | 100 | 90 | 90 |
| Ia-9 | 80 | g/ha | 90 | 90 | 90 | 90 | 80 | 90 | 90 | 90 | 90 | | 90 | 90 |
| Ia-3 | 80 | g/ha | 90 | 85 | 90 | | 80 | 85 | 90 | 90 | 90 | 90 | 80 | 80 |

In the table, the individual crops are abbreviated as follows:
ALOMY: Slender meadow foxtail (Alopecurus myosuroides)
AMARE: Redroot pigweed (Amaranthus retroflexus)
CHEAL: Lambs quarters (Chenopodium album)
DIGSA: Hairy crab grass (Digitaria sanguinalis)
ECHCG: Barnyard grass (Echinochloa crus-galls)
LOLMU: Italian rye grass (Lolium multiflorum)
MATIN: Scentless camomile (Matricaria inodora)
PHBPU: Purple morning glory (Pharbitis/Ipomoea purpurea)
SETVI: Green bristlegrass (Setaria viridis)
SINAL: White mustard (Sinapis alba)
STEME: Chickweed (Stellaria media)
VIOTR: Wild pansy (Viola tricolor)

### 2. Post-Emergence Herbicidal Activity

Seeds of monocotyledonous or dicotyledonous weed plants or crop plants are placed in sandy loam in wood fiber pots, covered with soil and grown in the greenhouse under good growth conditions. 2 to 3 weeks after sowing, the test plants are treated in the one-leaf stage. The compounds according to the invention, which are formulated as wettable powders (WP), are then sprayed onto the green plant parts in the form of an aqueous suspension or emulsion with a water application rate of 600 l/ha (converted) with addition of 0.2% of wetter. After the test plants have been left to stand in the greenhouse for approximately 3 weeks under optimal growth conditions, the activity of the preparation is scored visually in comparison with untreated controls (herbicidal activity in percent (%): 100% activity=plants have died, 0% activity=like control plants).

The following results were achieved when using the compounds according to the invention post-emergence:

| | Dosage | | | | | | | |
|---|---|---|---|---|---|---|---|---|
| Compound | rate | Unit | ABUTH | ALOMY | AMARE | CHEAL | ECHCG | LOLMU |
| Ic-1 | 80 | g/ha | 80 | 80 | 95 | 80 | 90 | 85 |
| Ia-2 | 80 | g/ha | 90 | 90 | 100 | 80 | 90 | 95 |
| Ia-9 | 80 | g/ha | 90 | 90 | 100 | 90 | 90 | 90 |
| Ia-3 | 80 | g/ha | 90 | 90 | 95 | 80 | 95 | 85 |
| Ia-8 | 80 | g/ha | | | 80 | | 80 | 80 |

| Compound | MATIN | PHBPU | POLCO | SETVI | STEME | VIOTR | XANST |
|---|---|---|---|---|---|---|---|
| Ic-1 | 90 | 90 | 90 | 85 | 90 | 90 | 90 |
| Ia-2 | 90 | 90 | 90 | 95 | 95 | 90 | 100 |
| Ia-9 | 100 | 100 | 90 | 80 | 90 | 90 | 100 |
| Ia-3 | 100 | 100 | 80 | 80 | 85 | 80 | 90 |
| Ia-8 | | | | | 80 | | |

In the table, the individual crops are abbreviated as follows:
ABUTH: Velvetleaf (Abutilon theophrasti)
ALOMY: Slender meadow foxtail (Alopecurus myosuroides)
AMARE: Redroot pigweed (Amaranthus retroflexus)
CHEAL: Lambs quarters (Chenopodium album)
ECHCG: Barnyard grass (Echinochloa crus-galls)
LOLMU: Italian rye grass (Lolium multiflorum)
MATIN: Scentless camomile (Matricaria inodora)
PHBPU: Purple morning glory (Pharbitis/Ipomoea purpurea)
POLCO: Black bindweed (Polygonum convolvulus)
SETVI: Green bristlegrass (Setaria viridis)
STEME: Chickweed (Stellaria media)
VIOTR: Wild pansy (Viola tricolor)
XANST: Rough cocklebear (Xanthium strumarium)

1. An N-azinyl-N′-pyridylsulfonylurea of the formula (I) 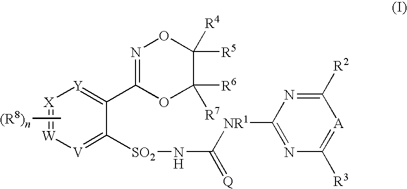
in which
W, X and Y are chosen such that one index of these indices represents a nitrogen atom and the remaining indices represent carbon atoms, the carbon atoms being unsubstituted or independently of one another substituted by R⁸;
V represents a carbon atom which is unsubstituted or substituted by R⁸;
is selected from the group consisting of nitrogen and CR⁹;
where
R⁹ is selected from the group consisting of hydrogen, alkyl, halogen and haloalkyl;
R¹ is selected from the group consisting of hydrogen and an unsubstituted or substituted radical from the series consisting of alkyl, alkoxy, alkoxyalkyl, alkenyl, alkynyl, cycloalkyl, cycloalkylalkyl, aralkyl and aryl;
R² is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino;
R³ is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino,
R⁴ to R⁷, in each case independently of one another, are selected from the group consisting of hydrogen, halogen, cyano, alkyl, alkoxy, alkylthio, alkylsulfinyl, alkylsulfonyl, alkylamino, dialkylamino, alkylcarbonyl, alkoxycarbonyl, alkylaminocarbonyl or dialkylaminocarbonyl, it being possible for the radicals to be unsubstituted or to have attached to them one or more radicals selected from the group consisting of halogen, cyano, alkoxy and alkylthio, or R⁴ and R⁶, and R⁵ and R⁷, respectively, represent an alkylidene group which is optionally interrupted by oxygen or sulfur,
R⁸ is selected from the group consisting of halogen, cyano, thiocyanato, nitro, alkyl, alkoxy, alkylthio, alkylsulfinyl, alkylsulfonyl, alkylamino, dialkylamino, alkylcarbonyl, alkoxycarbonyl, alkylaminocarbonyl, or dialkylaminocarbonyl, it being possible for the radicals to be unsubstituted or to have attached to them one or more radicals selected from the group consisting of halogen, cyano, alkoxy and alkylthio,
Q is selected from the group consisting of oxygen or sulfur,
n is an integer from 0 to 3,
and/or a salt of a compound of the formula (I),
with the proviso that, in those cases where n is 0, 1 or 2, unsubstituted carbon atoms V, W, X and/or Y are saturated with hydrogen.

2. An N-azinyl-N′-pyridylsulfonylurea as claimed in claim 1, wherein the substituent A is selected from the group consisting of nitrogen and CH.

3. An N-azinyl-N′-pyridylsulfonylurea as claimed in claim 1, wherein the substituent R¹ is selected from the group consisting of hydrogen, optionally halogen-substituted alkyl, optionally halogen-substituted alkoxy, optionally halogen-substituted alkoxyalkyl, optionally halogen-substituted alkenyl and optionally halogen-substituted alkynyl.

4. An N-azinyl-N′-pyridylsulfonylurea as claimed in claim 1, wherein the substituent R² is selected from the group consisting of hydrogen, halogen, optionally halogen-substituted alkyl, optionally halogen-substituted alkoxy, optionally halogen-substituted alkylthio, optionally halogen-substituted alkylamino and optionally halogen-substituted dialkylamino.

5. An N-azinyl-N′-pyridylsulfonylurea as claimed in claim 1, wherein the substituent R³ is selected from the group consisting of hydrogen, halogen, optionally halogen-substituted alkyl, optionally halogen-substituted alkoxy, optionally halogen-substituted alkylthio, optionally halogen-substituted alkylamino and optionally halogen-substituted dialkylamino.

6. An N-azinyl-N′-pyridylsulfonylurea as claimed in claim 1, wherein the substituents R⁴ to R⁷, in each case independently of one another, are selected from the group consisting of hydrogen, halogen, cyano, thiocyanato, optionally halogen-substituted alkyl, optionally halogen-substituted alkoxy, optionally halogen-substituted alkylthio, optionally halogen-substituted alkylsulfinyl, optionally halogen-substituted alkylsulfonyl, optionally halogen-substituted alkylamino, optionally halogen-substituted alkylcarbonyl, optionally halogen-substituted alkoxycarbonyl and optionally halogen-substituted alkylaminocarbonyl.

7. An N-azinyl-N′-pyridylsulfonylurea as claimed in claim 1, wherein the substituent R⁸ is selected from the group consisting of halogen, cyano, thiocyanato, optionally halogen-substituted alkyl, optionally halogen-substituted alkoxy, optionally halogen-substituted alkylthio, optionally halogen-substituted alkylsulfinyl, optionally halogen-substituted alkylsulfonyl, optionally halogen-substituted alkylamino, optionally halogen-substituted alkylcarbonyl, optionally halogen-substituted alkoxycarbonyl and optionally halogen-substituted alkylaminocarbonyl.

8. An N-azinyl-N′-pyridylsulfonylurea as claimed in claim 1, wherein n is 0.

9. A process for the preparation N-azinyl-N′-pyridylsulfonylureas as claimed in claim 1, comprising at least one of the following:
(a)
reacting a (5,6-dihydro-1,4,2-dioxazin-3-yl)pyridylsulfonamide of the formula (II) 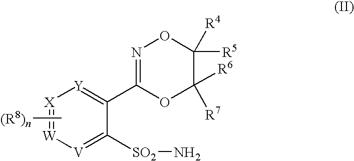
with a heterocyclic (thio)carbamate of the formula (III) 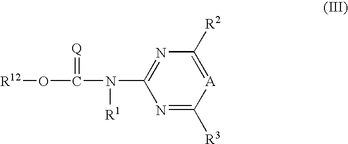
in which R¹² is a substituted or unsubstituted (C₁-C₂₀)-hydrocarbon radical optionally aryl, alkyl, substituted phenyl or optionally substituted (C₁-C₄)-alkyl;
or
(b)
reacting a 5,6-dihydro-1,4,2-dioxazin-3-ylpyridinesulfonyl iso(thio)cyanate of the formula (IV) 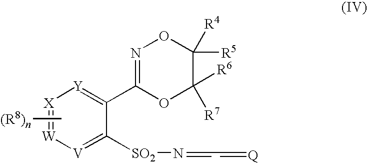
with an aminoheterocycle of the formula (V) 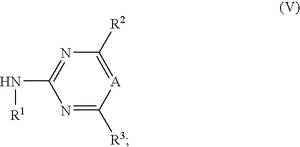
or
(c)
reacting a sulfonyl (thio)carbamate of the formula (VI) 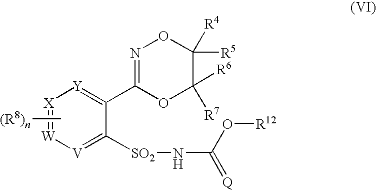
in which R¹² is a substituted or unsubstituted (C₁-C₂₀)-hydrocarbon radical optionally aryl, alkyl, substituted phenyl or optionally substituted (C₁-C₄)-alkyl, with an amino heterocycle of the formula (V) 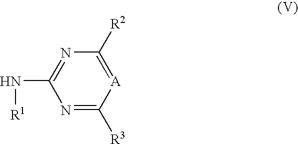
or
(d)
reacting a (5,6-dihydro-1,4,2-dioxazin-3-yl)pyridylsulfonamide of the formula (II) 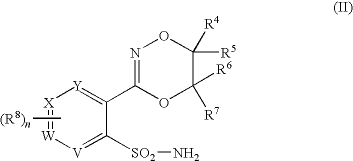
with an iso(thio)cyanate of the formula (VII) 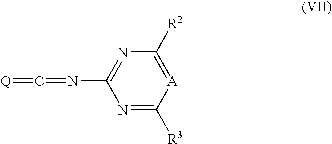
in which R¹ is hydrogen, optionally in the presence of a reaction auxiliary; or
(e)
initially reacting, with base catalysis, an amino heterocycle of the formula (V) 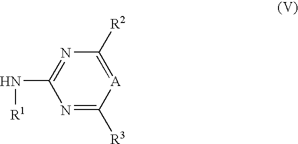
with a carbonic ester, which optionally comprises a diphenyl carbonate, and reacting, in a one-pot reaction, the resulting intermediate of the formula (III) 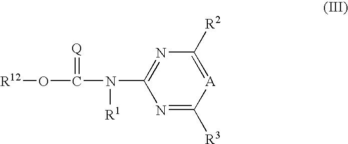
with a (5,6-dihydro-1,4,2-dioxazin-3-yl)pyridylsulfonamide of the formula (II) 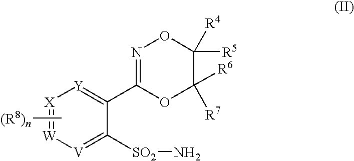
in which R¹ is hydrogen, or
(f)
reacting a (5,6-dihydro-1,4,2-dioxazin-3-yl)pyridylsulfonyl halide of the formula (VIII) 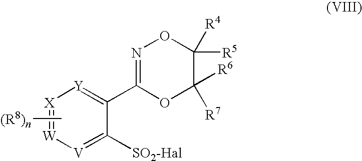
in which Hal is a halogen atom, with a (thio)cyanate which is optionally a metal (thio)cyanate, to give an iso(thio)cyanate of the formula (IV) 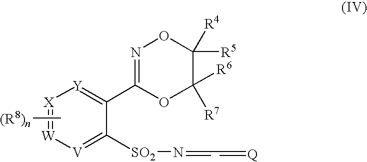
or a solvated (stabilized) derivative thereof, and subsequently with an amino heterocycle of the formula (V) 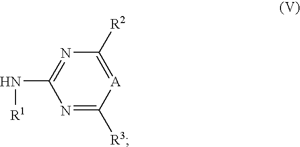
or
(g)
where Q=oxygen, reacting a (5,6-dihydro-1,4,2-dioxazin-3-yl)pyridylsulfonamide of the formula (II) 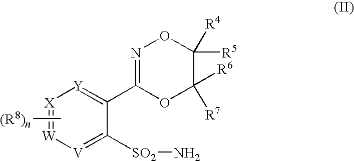
with a heterocyclic biscarbamate of the formula (IX) 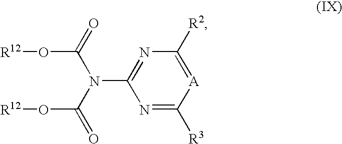
in which R¹² is a substituted or unsubstituted (C₁-C₂₀)-hydrocarbon radical, optionally aryl, alkyl, optionally substituted phenyl or optionally substituted (C₁-C₄)-alkyl, in the presence of a basic reaction auxiliary; or
(h)
initially reacting, with base catalysis, a (5,6-dihydro-1,4,2-dioxazin-3-yl)pyridylsulfonamide of the formula II 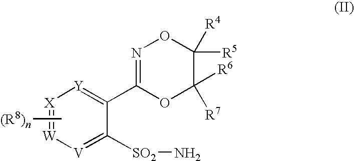
with a carbonic ester, which optionally comprises diphenyl carbonate, and reacting, in a one-pot reaction, the resulting intermediate of the formula (VI) 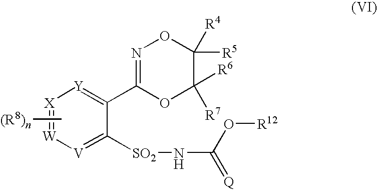
with an amino heterocycle of the formula (V).

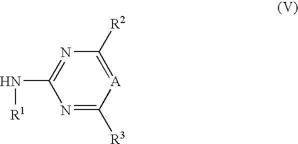

10. A compound of the formula (II) 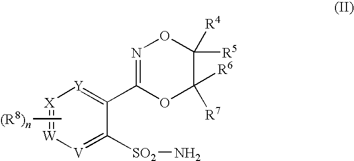
in which
W, X and Y are chosen such that one index of these indices represents a nitrogen atom and the remaining indices represent carbon atoms, the carbon atoms being unsubstituted or independently of one another substituted by R⁸;
V represents a carbon atom which is unsubstituted or substituted by R⁸;
A is selected from the group consisting of nitrogen and CR⁹;
where
R⁹ is selected from the group consisting of hydrogen, alkyl, halogen and haloalkyl;
R¹ is selected from the group consisting of hydrogen and an unsubstituted or substituted radical from the series consisting of alkyl, alkoxy, alkoxyalkyl, alkenyl, alkynyl, cycloalkyl, cycloalkylalkyl, aralkyl and aryl;
R² is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino;
R³ is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino,
R⁴ to R⁷, in each case independently of one another, are selected from the group consisting of hydrogen, halogen, cyano, alkyl, alkoxy, alkylthio, alkylsulfinyl, alkylsulfonyl, alkylamino, dialkylamino, alkylcarbonyl, alkoxycarbonyl, alkylaminocarbonyl or dialkylaminocarbonyl, it being possible for the radicals to be unsubstituted or to have attached to them one or more radicals selected from the group consisting of halogen, cyano, alkoxy and alkylthio, or R⁴ and R⁶, and R⁵ and R⁷, respectively, represent an alkylidene group which is optionally interrupted by oxygen or sulfur,
R⁸ is selected from the group consisting of halogen, cyano, thiocyanato, nitro, alkyl, alkoxy, alkylthio, alkylsulfinyl, alkylsulfonyl, alkylamino, dialkylamino, alkylcarbonyl, alkoxycarbonyl, alkylaminocarbonyl, or dialkylaminocarbonyl, it being possible for the radicals to be unsubstituted or to have attached to them one or more radicals selected from the group consisting of halogen, cyano, alkoxy and alkylthio,
Q is selected from the group consisting of oxygen or sulfur,
n is an integer from 0 to 3,
and/or a salt of a compound of the formula (I),
with the proviso that, in those cases where n is 0, 1 or 2, unsubstituted carbon atoms V, W, X and/or Y are saturated with hydrogen.

11. A compound of the formula (IV) 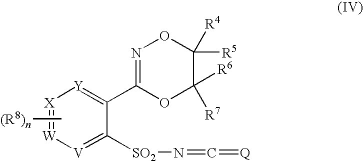
in which
W, X and Y are chosen such that one index of these indices represents a nitrogen atom and the remaining indices represent carbon atoms, the carbon atoms being unsubstituted or independently of one another substituted by R⁸;
V represents a carbon atom which is unsubstituted or substituted by R⁸;
A is selected from the group consisting of nitrogen and CR⁹;
where
R⁹ is selected from the group consisting of hydrogen, alkyl, halogen and haloalkyl;
R¹ is selected from the group consisting of hydrogen and an unsubstituted or substituted radical from the series consisting of alkyl, alkoxy, alkoxyalkyl, alkenyl, alkynyl, cycloalkyl, cycloalkylalkyl, aralkyl and aryl;
R² is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino;
R³ is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino,
R⁴ to R⁷, in each case independently of one another, are selected from the group consisting of hydrogen, halogen, cyano, alkyl, alkoxy, alkylthio, alkylsulfinyl, alkylsulfonyl, alkylamino, dialkylamino, alkylcarbonyl, alkoxycarbonyl, alkylaminocarbonyl or dialkylaminocarbonyl, it being possible for the radicals to be unsubstituted or to have attached to them one or more radicals selected from the group consisting of halogen, cyano, alkoxy and alkylthio, or R⁴ and R⁶, and R⁵ and R⁷, respectively, represent an alkylidene group which is optionally interrupted by oxygen or sulfur,
R⁸ is selected from the group consisting of halogen, cyano, thiocyanato, nitro, alkyl, alkoxy, alkylthio, alkylsulfinyl, alkylsulfonyl, alkylamino, dialkylamino, alkylcarbonyl, alkoxycarbonyl, alkylaminocarbonyl, or dialkylaminocarbonyl, it being possible for the radicals to be unsubstituted or to have attached to them one or more radicals selected from the group consisting of halogen, cyano, alkoxy and alkylthio,
Q is selected from the group consisting of oxygen or sulfur,
n is an integer from 0 to 3,
and/or a salt of a compound of the formula (I),
with the proviso that, in those cases where n is 0, 1 or 2, unsubstituted carbon atoms V, W, X and/or Y are saturated with hydrogen.

12. A compound of the formula (X) 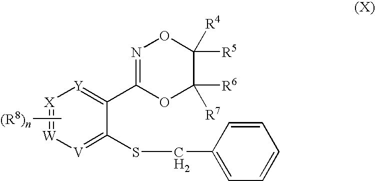
in which
W, X and Y are chosen such that one index of these indices represents a nitrogen atom and the remaining indices represent carbon atoms, the carbon atoms being unsubstituted or independently of one another substituted by R⁸;
V represents a carbon atom which is unsubstituted or substituted by R⁸;
A is selected from the group consisting of nitrogen and CR⁹;
where
R⁹ is selected from the group consisting of hydrogen, alkyl, halogen and haloalkyl;
R¹ is selected from the group consisting of hydrogen and an unsubstituted or substituted radical from the series consisting of alkyl, alkoxy, alkoxyalkyl, alkenyl, alkynyl, cycloalkyl, cycloalkylalkyl, aralkyl and aryl;
R² is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino;
R³ is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino,
R⁴ to R⁷, in each case independently of one another, are selected from the group consisting of hydrogen, halogen, cyano, alkyl, alkoxy, alkylthio, alkylsulfinyl, alkylsulfonyl, alkylamino, dialkylamino, alkylcarbonyl, alkoxycarbonyl, alkylaminocarbonyl or dialkylaminocarbonyl, it being possible for the radicals to be unsubstituted or to have attached to them one or more radicals selected from the group consisting of halogen, cyano, alkoxy and alkylthio, or R⁴ and R⁶, and R⁵ and R⁷, respectively, represent an alkylidene group which is optionally interrupted by oxygen or sulfur,
R⁸ is selected from the group consisting of halogen, cyano, thiocyanato, nitro, alkyl, alkoxy, alkylthio, alkylsulfinyl, alkylsulfonyl, alkylamino, dialkylamino, alkylcarbonyl, alkoxycarbonyl, alkylaminocarbonyl, or dialkylaminocarbonyl, it being possible for the radicals to be unsubstituted or to have attached to them one or more radicals selected from the group consisting of halogen, cyano, alkoxy and alkylthio,
Q is selected from the group consisting of oxygen or sulfur,
n is an integer from 0 to 3,
and/or a salt of a compound of the formula (I),
with the proviso that, in those cases where n is 0, 1 or 2, unsubstituted carbon atoms V, W, X and/or Y are saturated with hydrogen.

13. A compound of the formula (VIII) 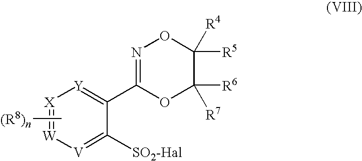
in which
W, X and Y are chosen such that one index of these indices represents a nitrogen atom and the remaining indices represent carbon atoms, the carbon atoms being unsubstituted or independently of one another substituted by R⁸;
V represents a carbon atom which is unsubstituted or substituted by R⁸;
A is selected from the group consisting of nitrogen and CR⁹;
where
R⁹ is selected from the group consisting of hydrogen, alkyl, halogen and haloalkyl;
R¹ is selected from the group consisting of hydrogen and an unsubstituted or substituted radical from the series consisting of alkyl, alkoxy, alkoxyalkyl, alkenyl, alkynyl, cycloalkyl, cycloalkylalkyl, aralkyl and aryl;
R² is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino;
R³ is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino,
R⁴ to R⁷, in each case independently of one another, are selected from the group consisting of hydrogen, halogen, cyano, alkyl, alkoxy, alkylthio, alkylsulfinyl, alkylsulfonyl, alkylamino, dialkylamino, alkylcarbonyl, alkoxycarbonyl, alkylaminocarbonyl or dialkylaminocarbonyl, it being possible for the radicals to be unsubstituted or to have attached to them one or more radicals selected from the group consisting of halogen, cyano, alkoxy and alkylthio, or R⁴ and R⁶, and R⁵ and R⁷, respectively, represent an alkylidene group which is optionally interrupted by oxygen or sulfur,
R⁸ is selected from the group consisting of halogen, cyano, thiocyanato, nitro, alkyl, alkoxy, alkylthio, alkylsulfinyl, alkylsulfonyl, alkylamino, dialkylamino, alkylcarbonyl, alkoxycarbonyl, alkylaminocarbonyl, or dialkylaminocarbonyl, it being possible for the radicals to be unsubstituted or to have attached to them one or more radicals selected from the group consisting of halogen, cyano, alkoxy and alkylthio,
Q is selected from the group consisting of oxygen or sulfur,
n is an integer from 0 to 3,
and/or a salt of a compound of the formula (I),
with the proviso that, in those cases where n is 0, 1 or 2, unsubstituted carbon atoms V, W, X and/or Y are saturated with hydrogen
and Hal represents a halogen atom.

14. A compound of the formula (VI) 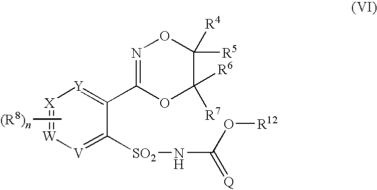
in which
W, X and Y are chosen such that one index of these indices represents a nitrogen atom and the remaining indices represent carbon atoms, the carbon atoms being unsubstituted or independently of one another substituted by R⁸;
V represents a carbon atom which is unsubstituted or substituted by R⁸;
A is selected from the group consisting of nitrogen and CR⁹;
where
R⁹ is selected from the group consisting of hydrogen, alkyl, halogen and haloalkyl;
R¹ is selected from the group consisting of hydrogen and an unsubstituted or substituted radical from the series consisting of alkyl, alkoxy, alkoxyalkyl, alkenyl, alkynyl, cycloalkyl, cycloalkylalkyl, aralkyl and aryl;
R² is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino;
R³ is selected from the group consisting of hydrogen, halogen, optionally halogen-substituted alkyl, optionally halogen-substituted alkoxy, optionally halogen-substituted alkylthio, optionally halogen-substituted alkylamino or optionally halogen-substituted dialkylamino,
R⁴ to R⁷, in each case independently of one another, are selected from the group consisting of hydrogen, halogen, cyano, alkyl, alkoxy, alkylthio, alkylsulfinyl, alkylsulfonyl, alkylamino, dialkylamino, alkylcarbonyl, alkoxycarbonyl, alkylaminocarbonyl or dialkylaminocarbonyl, it being possible for the radicals to be unsubstituted or to have attached to them one or more radicals selected from the group consisting of halogen, cyano, alkoxy and alkylthio, or R⁴ and R⁶, and R⁵ and R⁷, respectively, represent an alkylidene group which is optionally interrupted by oxygen or sulfur,
R⁸ is selected from the group consisting of halogen, cyano, thiocyanato, nitro, alkyl, alkoxy, alkylthio, alkylsulfinyl, alkylsulfonyl, alkylamino, dialkylamino, alkylcarbonyl, alkoxycarbonyl, alkylaminocarbonyl, or dialkylaminocarbonyl, it being possible for the radicals to be unsubstituted or to have attached to them one or more radicals selected from the group consisting of halogen, cyano, alkoxy and alkylthio,
Q is selected from the group consisting of oxygen or sulfur,
n is an integer from 0 to 3,
and/or a salt of a compound of the formula (I),
with the proviso that, in those cases where n is 0, 1 or 2, unsubstituted carbon atoms V, W, X and/or Y are saturated with hydrogen
and in which R¹² represents a substituted or unsubstituted (C₁-C₂₀)-hydrocarbon radical which is optionally aryl, alkyl, optionally substituted phenyl or optionally substituted (C₁-C₄)-alkyl.

15. A composition comprising at least one compound of the formula (I) according to claim 1.

16. The composition as claimed in claim 15, which comprises at least one further active substance which is selected from the group consisting of at least one further herbicide and at least one safener.

17. A herbicide and/or plant growth regulator comprising at least one compound of claim 1.

18. An herbicide and/or plant growth regulator comprising a composition of claim 15.

19. A method for controlling plants in specific plant crops and/or as plant growth regulator comprising applying a composition of claim 16 to plant control and/or to regulate plant growth.
